(19) United States
(12) Patent Application Publication
(10) Pub. No.: US 2022/0324796 A1
(43) Pub. Date: Oct. 13, 2022

(54) ANTIBIOTIC COMPOUNDS

(71) Applicants: The General Hospital Corporation, Boston, MA (US); Rhode Island Hospital, A Lifespan- Partner, Providence, RI (US); Emory Univerversity, Atlanta, GA (US)

(72) Inventors: Frederick M. Ausubel, Newton, MA (US); Wooseong Kim, Providence, RI (US); Eleftherios Mylonakis, Providence, RI (US); William M. Wuest, Atlanta, GA (US)

(21) Appl. No.: 17/618,333

(22) PCT Filed: Jun. 11, 2020
(86) PCT No.: PCT/US2020/037178

§ 371 Date: Dec. 10, 2021

Related U.S. Application Data
(60) Provisional application No. 62/860,475, filed on Jun. 12, 2019.

(51) Int. Cl. C07C 321/30; C07C 317/14; C07C 49/786; A61P 31/04

(52) U.S. Cl. CPC C07C 321/30; C07C 317/14; C07C 49/786; A61P 31/04

(57) ABSTRACT

The present application provides compounds and methods for treating bacterial infections, including bacterial infections caused by MRSA.

### CLAIM OF PRIORITY

This application claims priority to U.S. Provisional Patent Application Serial No. 62/860,475, filed on Jun. 12, 2019, the entire contents of which are hereby incorporated by reference.

### FEDERALLY SPONSORED RESEARCH OR DEVELOPMENT

This invention was made with Government support under Grant Nos. A1083214 and GM119426 awarded by the National Institutes of Health. The Government has certain rights in the invention.

### TECHNICAL FIELD

This invention relates to compounds useful in treating bacterial infections.

### BACKGROUND

Staphylococcus aureus and Enterococcus species have emerged as significant Gram-positive bacterial pathogens, presenting drug resistant strains such as methicillin resistant S. aureus (MRSA), vancomycin resistant S. aureus (VRSA), and vancomycin resistant Enterococcus (VRE). In 2005, 94,000 life-threating infections were attributed to S. aureus. Like MRSA, VRE has also become an important nosocomial pathogen, causing outbreaks in hospitals all over the world. VRE has been documented to colonize patients in dialysis units, neonatal units, hematology/oncology wards, and liver transplant units. The current arsenal of drugs is not sufficient to treat these infections.

### SUMMARY

Treatment of bacterial infections is complicated by the development of antibiotic resistance and tolerance, a consequence of the ability of bacterial pathogens to enter into non-growing, dormant states (persister states). Antibiotic-resistant bacteria are clinically responsible for chronic and relapsing infections. The compounds of the present disclosure efficiently kill bacterial persister cells, such as methicillin-resistant S. aureus (MRSA) persister cells. The compounds achieve this by rapid penetration and embedding in bacterial lipid bilayers, concomitant increase in the bacterial membrane fluidity and ultimate membrane disruption. What is more, the compounds are highly selective for bacterial lipid bilayer membranes versus cholesterol-rich mammalian membranes. Advantageously, the compounds disrupt bacterial membrane lipid bilayers at concentrations that exhibit low levels of toxicity to mammalian cells.

In one general aspect, the present disclosure provides a compound of Formula (I)

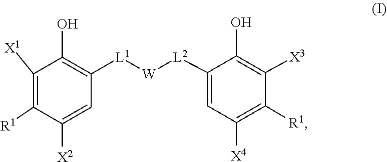

or a pharmaceutically acceptable salt thereof, wherein X¹, X², X³, X⁴, R¹, R², L¹, L², and W are as described herein.

In another general aspect, the present disclosure provides a pharmaceutical composition comprising a compound of Formula (I), or a pharmaceutically acceptable salt thereof, and a pharmaceutically acceptable carrier.

In yet another general aspect, the present disclosure provides a method of killing or inhibiting growth of a Gram-positive bacteria, the method comprising contacting the bacteria with an effective amount of a compound of Formula (I), or a pharmaceutically acceptable salt thereof.

In yet another general aspect, the present disclosure provides a method of treating a bacterial infection caused by Gram-positive bacteria, the method comprising administering to the subject in need thereof a therapeutically effective amount of a compound of Formula (I), or a pharmaceutically acceptable salt thereof, or a pharmaceutical composition comprising same.

In yet another general aspect, the present disclosure provides a method of killing or inhibiting growth of Gram-positive bacteria which is tolerant or resistant to one or more other antibiotic agents, the method comprising contacting the bacteria with an effective amount of a compound of formula:

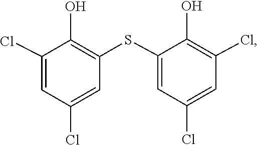 [SMILES: Oc1c(Cl)cc(Cl)cc1Sc1cc(Cl)cc(Cl)c1O]

or a pharmaceutically acceptable salt thereof.

In yet another general aspect, the present disclosure provides a method of treating a bacterial infection caused by Gram-positive bacteria which is tolerant or resistant to one or more other antibiotic agents, the method comprising administering to the subject in need thereof a therapeutically effective amount of a compound of formula:

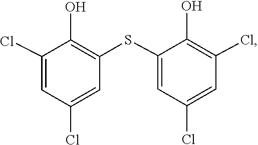 [SMILES: Oc1c(Cl)cc(Cl)cc1Sc1cc(Cl)cc(Cl)c1O]

or a pharmaceutically acceptable salt thereof.

In yet another general aspect, the present disclosure provides a compound of formula (1):

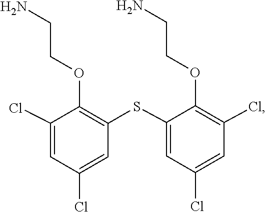 [SMILES: NCCOc1c(Cl)cc(Cl)cc1Sc1cc(Cl)cc(Cl)c1OCCN]

or a pharmaceutically acceptable salt thereof.

In yet another general aspect, the present disclosure provides a method of killing or inhibiting growth of bacteria, the method comprising contacting the bacteria with an effective amount of a compound formula (1), or a pharmaceutically acceptable salt thereof.

In yet another general aspect, the present disclosure provides a method of treating a bacterial infection the method comprising administering to the subject in need thereof a therapeutically effective amount of a compound of formula (1), or a pharmaceutically acceptable salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the present application will be apparent from the following detailed description and figures, and from the claims.

### DESCRIPTION OF DRAWINGS

FIGS. 1A-1E Bithionol shows bactericidal activity against S. aureus persisters. (A) Chemical structures of bithionol. (B) TEM micrographs showing the formation of intracellular mesosome-like structures, an abnormal cell division, or cell lysis in S. aureus strain MW2 treated with 10× MIC (10 μg/mL) bithionol. S. aureus MW2 was treated with 10× MIC bithionol or 0.1% DMSO (control) for 2 h. Scale bars represent 500 nm. (C, D) Viability of MRSA MW2 stationary-phase (C) or biofilm (D) persister cells treated with 100× MIC of the conventional antibiotics vancomycin (Van), gentamicin (Gm), ciprofloxacin (Cipro), daptomycin (Dap), linezolid (Lin), or the indicated concentrations of bithionol (BT) for 4 h (C) and 24 h (D), respectively. (E) The viability of S. aureus VRS1 persisters treated with 100× MIC (200 μg/mL) linezolid (Lin), 100× MIC (100 μg/mL) daptomycin (dap), or the indicated concentrations of bithionol (BT) as a function of time. The data points on the x-axis are below the level of detection (2×10² CFU/mL, or 2×10² CFU/membrane). Individual data points (n=3 biologically independent samples) are shown; error bars represent means±s.d.

FIGS. 2A-2E. Bithionol selectively disrupts bacterial lipid bilayers. (A) Representative configurations of MD simulations of bithionol from left to right, onset, membrane attachment, membrane penetration, and equilibrium interacting with 7DOPC/3DOPG or 7POPC/3cholesterol lipid bilayers. Bithionol and sodium ions are depicted as large spheres, phospholipids are represented as chains, and bonds in cholesterols are highlighted by thickened tubes. Water molecules are set to be transparent for clarity. (B) The free energy profiles of bithionol penetrating into the indicated lipid bilayers as a function of the center-of-mass (COM) distance to the bilayer. The dot-dashed lines mark the surface of bacterial and mammalian membranes, respectively, averaged from the COM locations of phosphate groups in the lipids of the outer leaflets. Error bars represent means±s.d. from three independent simulations. (C) GUVs consisting of DOPC/DOPG (7:3) or POPC/cholesterol (7:3) labeled with 0.005% Liss Rhod PE were treated with the indicated concentrations of bithionol or DMSO (control) and were monitored over time using fluorescence microscopy. Scale bars represent 10 μm. (I)) Uptake of SYTOX Green (Ex=485 nm, Em=525 nm) by MRSA MW2 persister cells or human renal proximal cells (HKC-8) treated with the indicated concentrations of bithionol. Results are shown as means; n=3 biologically independent samples. Error bars not shown for clarity. (E) S. aureus MW2 membrane fluidity treated with the indicated concentrations of bithionol for 1 h was evaluated based on Laurdan generalized polarization (Laurdan GP). Laurdan GP=(I₄₄₀−I₄₉₀)/(I₄₄₀+I₄₉₀) where I₄₄₀ and I₄₉₀ are the emission intensities at 440 and 490 nm, respectively when excited at 350 nm. The membrane fluidizer benzyl alcohol (50 mM) was used as a positive control. Individual data points (n=3 biologically independent samples) are shown; error bars represent means±s.d. Statistical differences between control and antibiotic treatment groups were analyzed by one-way ANOVA and post-hoc Dunnett test (**p=0.01, ***p<0.0001.)

FIGS. 3A-3C. Bithionol shows synergism with gentamicin against MRSA persisters, in vitro and in vivo. (A) Stationary phase or (B) biofilm MRSA MW2 persisters were treated with the indicated concentrations of bithionol combined with gentamicin (Gm). Colony forming units (CFUs) were measured by serial dilution and plating on TSA plates. The data points on the x-axis are below the level of detection (2×10² CFU/mL, or 2×10² CFU/membrane). Individual data points (n=3 biologically independent samples) are shown; error bars represent means±s.d. (C) Ten infected mice per group (n=10 biologically independent animals) were treated with control (5% Killophor+5% ethanol, i.p.), vancomycin (30 mg/kg, i.p.), gentamicin (30 mg/kg, s.c.), bithionol (30 mg/kg, i.p.), vancomycin (30 mg/kg, i.p.), or bithionol (30 mg/kg, i.p.) combined with gentamicin (30 mg/kg, s.c.) every 12 h for 3 days at 24 h post-infection. At 12 h after the last treatment, mice were euthanized. Their thighs were excised and homogenized. CFUs from each mouse thigh are plotted as individual points and error bars represent the standard deviation in each experimental group. Statistical differences between control and antibiotic treatment groups were analyzed by one-way ANOVA and post-hoc Tukey test (***p<0.001).

FIGS. 4A-4B. Relationship between anti-persister activity and alternation in membrane fluidity. Membrane fluidity of (A) bithionol and its analogs or (B) nTZDpa and its analogs at 32 μg/mL was evaluated by Laurdan GP. The 50 mM of membrane fluidizer benzyl alcohol (B.A.) was used as positive control. (B) The numbers above each bar indicate persister killing concentration (PKC, μg/mL) to kill 5×10′ CFU/mL MRSA persister below the limit of detection (2×10² CFU/mL). (A, B) Individual data points (n=3 biologically independent experiments) are shown; error bars represent means±s.d. Statistical differences between control and antibiotic treatment groups were analyzed by one-way ANOVA and post-hoc Dunnett test (ns=p>0.05 no significance, *p=0.05, **p=0.01, ***p<0.001). Individual data points (n=3 biologically independent experiments) are shown; error bars represent means±s.d.

FIG. 5 contains a table showing the structure-activity relationships for antibiotic activity and membrane activity of the selected exemplified compounds. FIG. 6 contains a table showing minimum inhibitory concentration (μg/mL) of bithionol and various traditional antibiotics for a variety of bacterial strains.

FIG. 7 contains a table showing characteristic parameters derived from the energy profile of interaction between bithionol or exemplified compounds and the bacterial mimetic lipid bilayer.

FIGS. 8A-8C. Bithionol shows fast-killing kinetics and causes cell lysis. (A)

The viability of exponential phase MRSA MW2 cells treated with 10× MIC (10 μg/mL) bithionol, 10× MIC (10 μg/mL) daptomycin, 10× MIC (10 μg/mL) vancomycin as a function of time. The data points on the x-axis are below the level of detection (2×10² CFU/mL). (B) Exponential phase MRSA MW2 cells (OD_{600˜0.45}) were treated with 10× MIC bithionol, 10× MIC (20 μg/mL) Benzyldimethylhexadecylammonium chloride (16-BAC), or 10× MIC (1.25 μg/mL) nigericin for 4 h. The anti-infective detergent BAC was a control for cell lytic activity; the ionophore nigericin was used a control for bacteriostatic activity. Individual data points (n=3 biologically independent samples) are shown; error bars represent means±s.d. (C) Uptake of SYTOX Green (Ex=485 nm, Em=525 nm) by MRSA MW2 growing cells or persister cells treated with the indicated concentrations of bithionol or daptomycin. Results are shown as means; n=3 biologically independent samples. Error bars not shown for clarity.

FIGS. 9A-9C. (A) Detailed configurations of nearest neighboring lipids around an embedded bithionol molecule. Bithionol is described as large spheres. Phospholipids before and after the insertion of bithionol (1 nm around bithionol) are shown as chains of atoms. Water molecules are set to be transparent for clarity. (B) Normalized distances of bithionol from the bilayer centers of the indicated lipid bilayers versus simulation time. The distances from bilayer centers are normalized by half the thickness of the indicated lipid bilayers, respectively. (C) Change in lipid bilayer thickness after one bithionol molecule embeds into the outer leaflet of the bilayer. Bilayer thickness is obtained by time average over 50 ns simulation trajectory at equilibrium.

FIGS. 10A-10D. The effect of cholesterol in simulated mammalian membranes on bithionol penetration. (A) The free energy profile of bithionol penetrating into a POPC lipid bilayer with different molar percentages of cholesterols as a function of the COM distance to the bilayer. Error bars represent s.d. from three independent simulations. (B) Calculated mass density profile of the hydrophobic region (acyl chains) of POPC lipid bilayers containing different molar percentages of cholesterol versus the COM distance to the bilayer. (C) Calculated lateral mean squared displacements (MSD) of POPC lipids over 100 ns simulations at equilibrium for various molar percentages of cholesterol. MSD represents the deviation of the position of the molecules with respect to a reference position over time, which is a measurement of their diffusion coefficients. (D) Calculated deuterium order parameters (-S_{CD}) for saturated sn-1 and unsaturated sn-2 acyl chains in POPC lipids with different molar percentages of cholesterols. The deuterium order parameter represents the structural orientation or mobility of lipids in a bilayer, corresponding to the configurational entropy and the physical state (liquid-disordered or solid-ordered phases) of the membrane system. In general, a higher-S_{CD} indicates a higher ordered structure of lipids with decreased membrane fluidity and permeability.

FIGS. 11A-11B. All-atom molecular dynamics simulations using the CHARMM force field. (A) Representative configurations of MD simulations of bithionol interacting with 7DOPC/3DOPG or 7POPC/3 cholesterol lipid bilayers. (B) The free energy profiles of bithionol penetrating into the indicated lipid bilayers as a function of the center-of-mass (COM) distance to the bilayer.

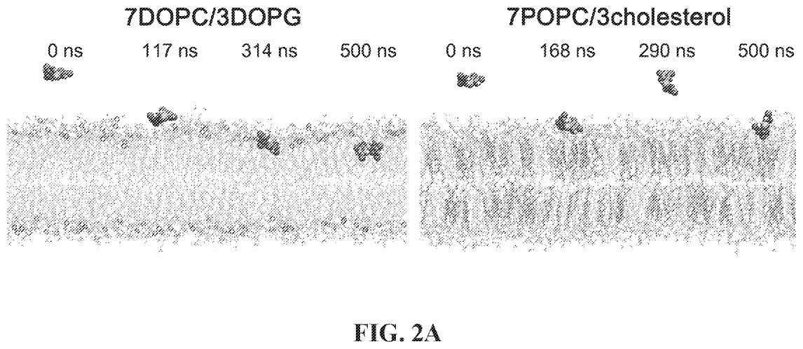

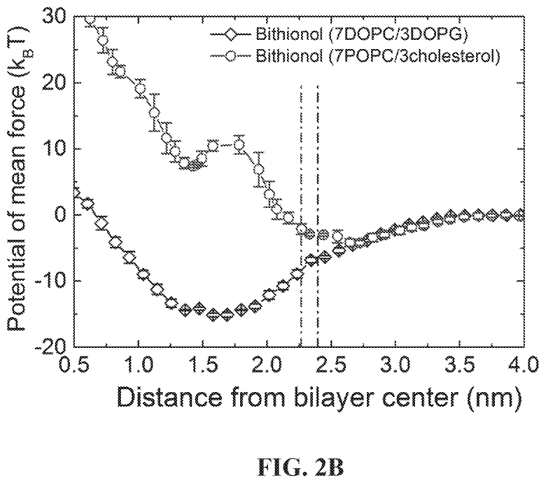

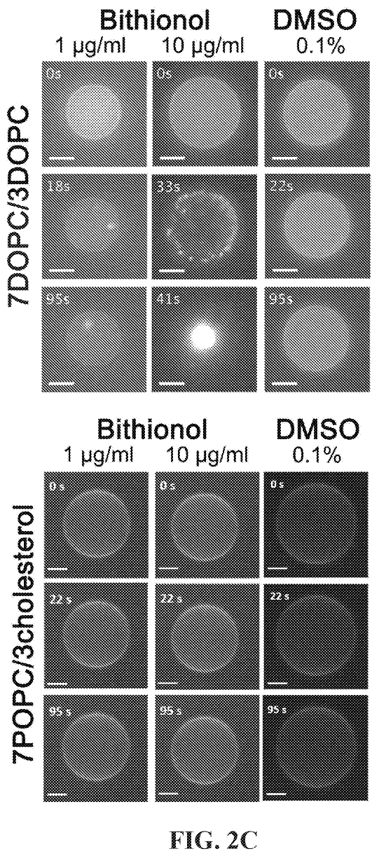

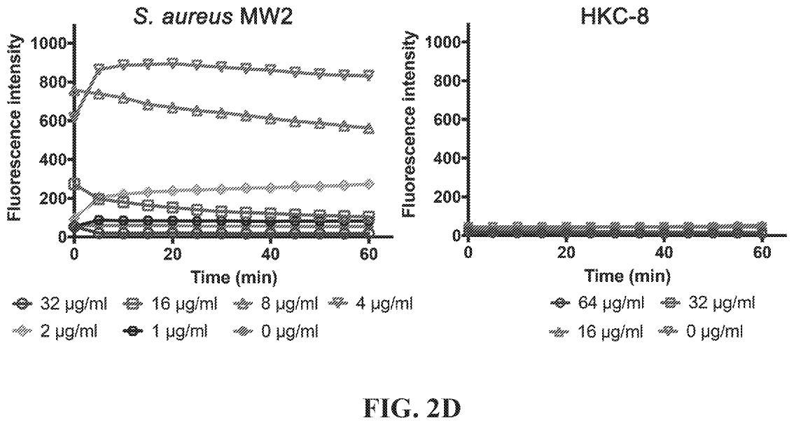

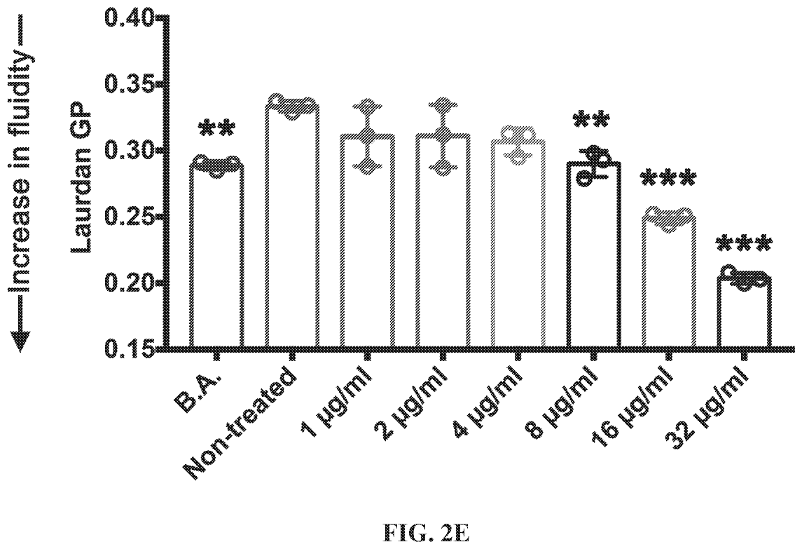

FIGS. 12A-12C. Bithionol exhibits relatively modest membrane activity against mammalian lipid bilayer membranes. (A) 2% human erythrocytes were treated with two-fold serially diluted concentrations of bithionol for 1 h at 37° C. A sample treated with 1% Triton-X 100 was used as the control for 100% hemolysis. Individual data points (n=3 biologically independent experiments) are shown; error bars represent means±s.d. (B) A human renal proximal cell line, HKC-8, was treated with the indicated concentration of a cholesterol binding detergent, saponin (positive control for FIG. 2D). Membrane permeability was evaluated spectrophotometrically by monitoring the uptake of SYTOX Green (Ex=485 nm, Em=525 nm) for 1 h. Results are shown as means; n=3 biologically independent samples. Error bars not shown for clarity. (C) HKC-8 cells were treated with 32 μg/mL bithionol or 32 μg/mL saponin for 1 h. The HKC-8 cells were stained with 1 μM SYTOX Green, which only stains the cells having compromised membranes. Bright field and fluorescent images (Ex=470/40 nm, Em=525/40 nm) were obtained using a Zeiss Axiovert 200 microscope. The scale bar represents 1μm.

FIG. 13. Effects of sulfoxide or methoxy analogs of bithionol on MRSA membrane permeability, membrane fluidity and persister viability. Membrane permeability was determined based on uptake of SYTOX Green by MRSA MW2 cells. Results are shown as means; n=3 biologically independent samples. Error bars not shown for clarity. Membrane fluidity was evaluated based on Laurdan GP. The 50 mM of membrane fluidizer benzyl alcohol (B.A.) was used as positive control. Individual data points (n=3 biologically independent experiments) are shown; error bars represent means±s.d. Statistical differences between control and antibiotic treatment groups were analyzed by one-way ANOVA and post-hoc Dunnett test (*p=0.05, ***p<0.001). Viability of MRSA MW2 persister cells was determined after the treatment with the indicated concentration of bithionol analogs for 4h. The data points on the x-axis are below the level of detection (2×10² CFU/mL). Individual data points (n=3 biologically independent experiments) are shown; error bars represent means±s.d.

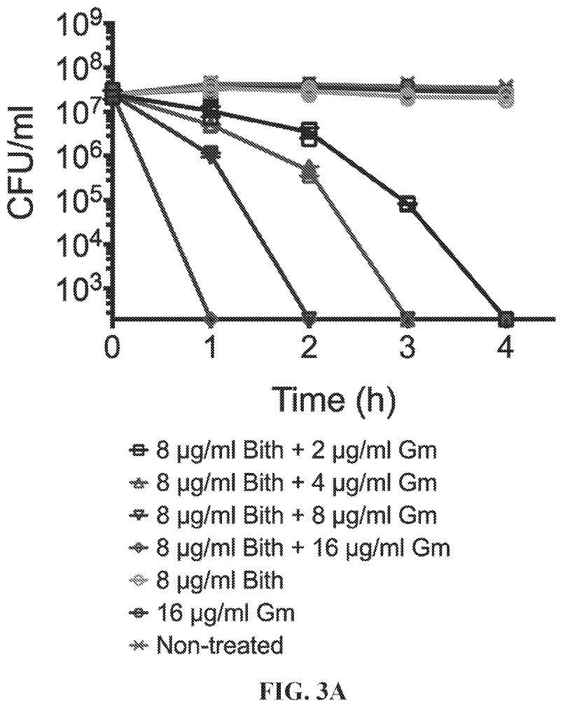

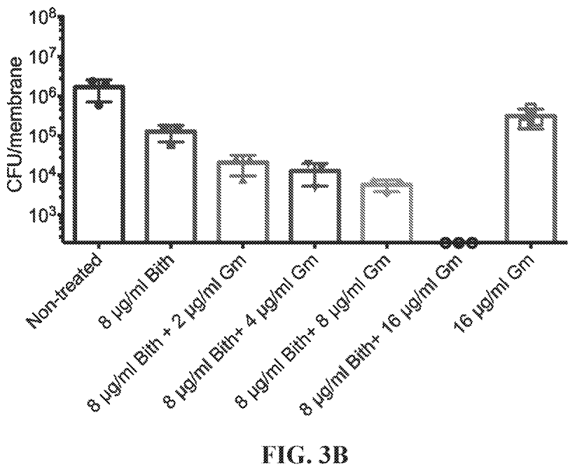

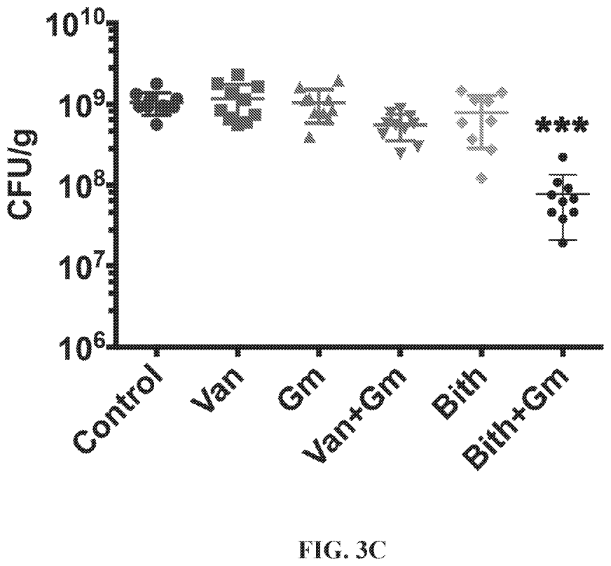

FIG. 14. Effects of halogen analogs of bithionol on MRSA membrane permeability, membrane fluidity and persister viability. Membrane permeability was determined based on uptake of SYTOX Green by MRSA MW2 cells. Results are shown as means; n=3 biologically independent samples. Error bars not shown for clarity. Membrane fluidity was evaluated by Laurdan GP. The 50 mM of membrane fluidizer benzyl alcohol (B.A.) was used as positive control. Individual data points (n=3 biologically independent experiments) are shown; error bars represent means±s.d. Statistical differences between control and antibiotic treatment groups were analyzed by one-way ANOVA and post-hoc Dunnett test (*p=0.05, **p=0.01, ***p<0.001). Viability of MRSA MW2 persister cells was determined after the treatment with the indicated concentration of bithionol analogs for 4h. The data points on the x-axis are below the level of detection (2×10² CFU/ml). Individual data points (n=3 biologically independent experiments) are shown; error bars represent means±s.d. FIGS. 15A-15B. Bithionol alone or bithionol combined with gentamicin shows no signs of hepatic or renal toxicity. Ten infected mice per group were treated with control (5% Killophor +5% ethanol, i.p.), vancomycin (30 mg/kg, i.p.), gentamicin (30 mg/kg, s.c.), bithionol (30 mg/kg, i.p.), or a vancomycin (30 mg/kg, i.p.) or bithionol (30 mg/kg, i.p.) combined with gentamicin (30 mg/kg, s.c.) every 12 h for 3 days at 24 h post-infection. At 12 h after the last treatment, mice were euthanized. Before excising thighs to evaluate bacterial loads (FIG. 3C), blood was collected and analyzed for ALT and BUN. International Units per Liter (IU/L) of ALT for each mouse serum (A) and absorbance at 340 nm of BUN (B) are plotted as individual points and error bars represent the deviation in each experimental group. Statistical differences between control and antibiotic treatment groups were analyzed by one-way ANOVA and post-hoc Tukey test (**p<0.01).

FIG. 16. An increase in membrane fluidity correlates with anti-persister potency. PKCs (μg/mL) and Laurdan GPs of bithionol, nTZDpa, and all their analogs at 32 pg/mL were plotted on the x and y axis, respectively. PKC >64 was regarded as 128. R-squared value and p value of slope were determined by Prism 7 software.

FIG. 17 contains a synthetic scheme showing chemical synthesis of exemplified compounds, and chemical structures of exemplified compounds.

FIG. 18 contains a synthetic scheme showing synthesis of compound BT-OMe.

### DETAILED DESCRIPTION

Staphylococcus aureus is a Gram-positive opportunistic human pathogen carried by approximately one third of the human population. Despite antibiotic availability, S. aureus infections are often hard-to-cure and remain one of the major causes of death. The failure of antibiotic therapy for S. aureus infections results both from the development of antibiotic-resistance as well as from the ability of S. aureus to enter into a non-growing antibiotic-tolerant state, referred to as persisters. Persisters show significantly reduced biosynthetic processes, which are the major targets for most current antibiotics. They also exist in a metabolically low-energy state that prevents the energy-dependent uptake of antibiotics such as aminoglycosides. S. aureus readily forms persisters, which are present in high numbers in stationary-phase suspension cultures and biofilms. These persisters are responsible for chronic and relapsing infections such as endocarditis, osteomyelitis, and prosthetic implant infections. Bacterial membranes are attractive anti-persister targets because they can be disrupted independently of growth. However, membrane-active agents are typically toxic to mammals due to low membrane selectivity. The clinical success of daptomycin, however, draws attention to membrane-active antimicrobial therapeutics. The lipophilic tail of daptomycin, a natural cyclic lipopeptide synthesized by Streptomyces roseosporus, is thought to insert into Gram-positive bacterial membranes and form oligomeric pores, thereby causing membrane depolarization, potassium ion efflux, and rapid cell death. Despite strong antimicrobial potency against growing bacterial cells, daptomycin has not been reported to be effective against persisters. In contrast to daptomycin, some previously reported synthetic membrane-targeting antibiotics not only kill the growing bacterial cells, but are also highly efficacious in killing and the non-growing MRSA persister cells. Like daptomycin, these membrane-active antimicrobial compounds insert into Gram-positive bacterial membranes causing rapid permeabilization and cell death. However, to be successful antibiotic therapeutics, these membrane-active compounds should also be relatively non-toxic and should exhibit a significant amount of selectivity for Gram-positive bacterial membranes compared to mammalian membranes. Hence, not all membrane-active compounds can be developed as anti-MRSA therapeutics. Some previously reported membrane-active compounds, for example, cause substantial hemolysis of red blood cells at high concentrations (over 32 μg/mL) despite excellent anti-MRSA persister activity. Advantageously, the compounds described in this application selectively attack bacterial compared to mammalian membrane lipid bilayers, e.g., due to lipid composition differences. The compounds, compositions containing these compounds, methods of using these compounds and compositions as antibacterial and bacteriostatic agents, and combination therapies containing these compounds, are described herein.

Antibacterial Compounds

In some embodiments, the present application provides a compound of Formula (I):

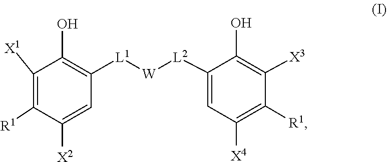

or a pharmaceutically acceptable salt thereof, wherein X¹, X², X³, X⁴, R¹, R², L¹, L², and W are as described herein.

In some embodiments:

W is selected from S, S(═O), S(═O)₂, O, and C(═O);

L¹ is selected from C₁₋₃ alkylene, C₂₋₄ alkenylene, C₂₋₄ alkynylene, and C₃₋₅ cycloalkylene, each of which is optionally substituted with 1, 2, or 3 substituents independently selected from OH, NH₂, NO₂, CN, and halo; or L¹ is absent;

L² is selected from C₁₋₃ alkylene, C₂₋₄ alkenylene, C₂₋₄ alkynylene, and C₃₋₅ cycloalkylene, each of which is optionally substituted with 1, 2, or 3 substituents independently selected from OH, NH₂, NO₂, CN, and halo; or L² is absent;

X¹, X², X³, and X⁴ are each independently selected from halo, Cy^{A}, CN, NO₂, C₁₋₆ alkyl, C₁₋₆ haloalkyl, C₂₋₆ alkenyl, C₂₋₆ alkynyl, SR^{a1}, S(O)R^{b1}, S(O)₂R^{b1}, and OR^{a1}; wherein said C₁₋₆ alkyl, C₂₋₆ alkenyl, and C₂₋₆ alkynyl are each optionally substituted with 1, 2, or 3 substituents independently selected from Cy^{A}, halo, CN, NO₂, OR^{a1}, SR^{al}, C(O)NR^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{cl}R^{d1}, S(O)₂R^{b1}, and S(O)₂NR^{c1}R^{d1};

each Cy^{A} is independently selected from C₆₋₁₀ aryl, C₃₋₁₀ cycloalkyl, 5-10 membered heteroaryl, and 4-10 membered heterocycloalkyl, each of which is optionally substituted with 1, 2, or 3 substituents independently selected from R^{Cy};

each R^{Cy} is independently selected from halo, C₁₋₄ alkyl, C₁₋₄ haloalkyl, C₂₋₆ alkenyl, C₂₋₆ alkynyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R¹ and R² are each independently selected from H, halo, CN, NO₂, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

each R^{a1}, R^{b1}, R^{c1}, R^{d1}, R^{a2} R^{b2}, R^{c2} and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted with 1, 2, or 3 substituents independently selected from R^{g};

or any R^{c1} and R^{d1} together with the N atom to which they are attached form a 4-, 5-, 6-, or 7-membered heterocycloalkyl group optionally substituted with 1, 2, or 3 substituents independently selected from R^{g};

or any R^{c2} and R^{d2} together with the N atom to which they are attached form a 4-, 5-, 6-, or 7-membered heterocycloalkyl group optionally substituted with 1, 2, or 3 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO-C₁₋₃ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di (C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the compound of Formula (I) is not any one of the following compounds:

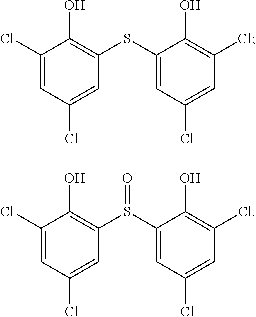

In some embodiments, the compound of Formula (I) is not the following compound:

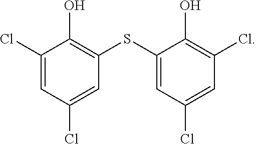 [SMILES: Oc1c(Cl)cc(Cl)cc1Sc1cc(Cl)cc(Cl)c1O]

In some embodiments, W is S. In some embodiments, W is S(═O). In some embodiments, W is O. In some embodiments, W is C(═O).

In some embodiments, L¹ is selected from C₁₋₃ alkylene, optionally substituted with OH, NH₂, NO₂, CN, and halo. In some embodiments, L¹ is C₁₋₃ alkylene substituted with OH. In some embodiments, L¹ is absent.

In some embodiments, L² is selected from C₁₋₃ alkylene, optionally substituted with OH, NH₂, NO₂, CN, and halo. In some embodiments, L² is C₁₋₃ alkylene substituted with OH. In some embodiments, L² is absent.

In some embodiments:

L¹ is selected from C₁₋₃ alkylene, C₂₋₄ alkenylene, C₂₋₄ alkynylene, and C₃₋₅ cycloalkylene, each of which is optionally substituted with 1, 2, or 3 substituents independently selected from OH, NH₂, NO₂, CN, and halo; and

L² is absent.

In some embodiments:

L¹ is absent; and

L² is selected from C₁₋₃ alkylene, C₂₋₄ alkenylene, C₂₋₄ alkynylene, and C₃₋₅ cycloalkylene, each of which is optionally substituted with 1, 2, or 3 substituents independently selected from OH, NH₂, NO₂, CN, and halo.

In some embodiments:

L¹ is C₁₋₃ alkylene substituted with OH; and

L² is absent.

In some embodiments:

L¹ is absent; and

L² is C₁₋₃ alkylene substituted with OH.

In some embodiments, L¹ is absent and L² is absent. In some embodiments, L¹ is C₁₋₃ alkylene and L² is C₁₋₃ alkylene. In some embodiments, L¹ is C₁₋₃ alkylene or absent, and L² is C₁₋₃ alkylene or absent.

In some embodiments, X¹, X², X³, and X⁴ are each independently selected from halo, Cy^{A}, CN, NO₂, C₁₋₆ alkyl, C₁₋₆ haloalkyl, and OR^{a1}; wherein said C₁₋₆ alkyl is optionally substituted with 1 or 2 substituents independently selected from Cy^{A}, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{c1}.

In some embodiments, X¹, X², X³, and X⁴ are each independently selected from halo, Cy^{A}, C₁₋₆ alkyl, and C₁₋₆ haloalkyl, wherein said C₁₋₆ alkyl is optionally substituted with a substituent selected from Cy^{A}, OH, NO₂, CN, halo, C₁₋₆ alkoxy, C₁₋₆ haloalkoxy, amino, C₁₋₆ alkylamino, and di(C₁₋₆ alkyl)amino

In some embodiments, X¹, X², X³, and X⁴ are each independently selected from halo, Cy^{A}, CN, NO₂, C₁₋₆ alkyl, C₁₋₆ haloalkyl, and OR^{a1}.

In some embodiments, at least one of X¹, X², X³, and X⁴ is selected from Br, Cy^{A}, C₁₋₆ alkyl, and C₁₋₆ haloalkyl, wherein said C₁₋₆ alkyl is optionally substituted with a substituent selected from Cy^{A}, OH, NO₂, CN, halo, C₁₋₆ alkoxy, C₁₋₆ haloalkoxy, amino, C₁₋₆ alkylamino, and di(C₁₋₆ alkyl)amino.

In some embodiments, at least one of X¹, X², X³, and X⁴ is selected from Cy^{A}, C₁₋₆ alkyl, and C₁₋₆ haloalkyl, wherein said C₁₋₆ alkyl is optionally substituted with a substituent selected from Cy^{A}, OH, NO₂, CN, halo, C₁₋₆ alkoxy, C₁₋₆ haloalkoxy, amino, C₁₋₆ alkylamino, and di(C₁₋₆ alkyl)amino.

In some embodiments, at least one of X¹, X², X³, and X⁴ is Cy^{A}.

In some embodiments, at least one of X¹, X², X³, and X⁴ is C₁₋₆ alkyl or C₁₋₆ haloalkyl.

In some embodiments, at least one of X¹, X², X³, and X⁴ is C₁₋₆ alkyl, optionally substituted with a substituent selected from Cy^{A}, OH, NO₂, CN, halo, C₁₋₆ alkoxy, C₁₋₆ haloalkoxy, amino, C₁₋₆ alkylamino, and di(C₁₋₆ alkyl)amino.

In some embodiments, at least one of X¹, X², X³, and X⁴ is C₁₋₆ alkyl, optionally substituted with Cy^{A}.

In some embodiments, X¹, X², X³, and X⁴ are each halo. In some embodiments, at least one of X¹, X², X³, and X⁴ is Br.

In some embodiments:

X¹ and X³ are each independently Br or F, and

X² and X⁴ are each independently Cl, F, or Br.

In some embodiments:

X¹ and X³ are each independently Cl, F, or Br, and

X² and X⁴ are each independently Br or F.

In some embodiments, each Cy^{A} is independently an C₆₋₁₀ aryl, optionally substituted with 1, 2, or 3 substituents independently selected from It^{c}Y. In some embodiments, each Cy^{A} is independently a phenyl, optionally substituted with 1, 2, or 3 substituents independently selected from R^{Cy}. In some embodiments, each Cy^{A} is independently a phenyl, optionally substituted with 1 or 2 substituents independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino.

In some embodiments, each Cy^{A} is independently a C₃₋₁₀ cycloalkyl, optionally substituted with 1, 2, or 3 substituents independently selected from R^{Cy}.

In some embodiments, each Cy^{A} is independently a 5-10 membered heteroaryl, optionally substituted with 1, 2, or 3 substituents independently selected from R^{Cy}.

In some embodiments, each Cy^{A} is independently a 4-10 membered heterocycloalkyl, optionally substituted with 1, 2, or 3 substituents independently selected from R^{Cy}.

In some embodiments, each RC' is independently selected from halo, C₁₋₄ alkyl, C₁₋₄ haloalkyl, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}C(O)NR^{c1}R^{dl}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, each RC' is independently selected from halo, OH, NO₂, CN, C₁₋₄ alkyl, C₁₋₄ haloalkyl, C₁₋₆ alkoxy, C₁₋₆ haloalkoxy, amino, C₁₋₆ alkylamino, and di(C₁₋₆alkyl)amino.

In some embodiments, le and R² are each independently selected from H, halo, CN, NO₂, OH C₁₋₆ alkyl, C₁₋₆ haloalkyl, C₁₋₆ alkoxy, C₁₋₆ haloalkoxy, amino, C₁₋₆ alkylamino, and di(C₁₋₆alkyl)amino.

In some embodiments, le and R² are each independently selected from H, halo, CN, NO₂, C₁₋₆ alkyl, C₁₋₆ haloalkyl, C₁₋₆ alkoxy, and C₁₋₆ haloalkoxy.

In some embodiments, each R^{a1}, R^{b1}, R^{c1}, R^{d1}, R^{a2}, R^{b2}, R^{c2}, and R^{d2} is independently selected from H, C₁₋₆ alkyl, and C₁₋₄ haloalkyl, wherein said C₁₋₆ alkyl is optionally substituted with 1 or 2 substituents independently selected from R^{g}.

In some embodiments, each R^{g} is independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino.

In some embodiments:

W is selected from S, S(═O), and O;

L¹ is C₁₋₃ alkylene or absent;

L² is C₁₋₃ alkylene or absent;

X¹, X², X³, and X⁴ are each independently selected from halo, Cy^{A}, C₁₋₆ alkyl, and C₁₋₆ haloalkyl, wherein said C₁₋₆ alkyl is optionally substituted a substituent selected from Cy^{A}, OH, NO₂, CN, halo, C₁₋₆ alkoxy, C₁₋₆ haloalkoxy, amino, C₁₋₆ alkylamino, and di(C₁₋₆alkyl)amino;

each Cy^{A} is independently a phenyl, optionally substituted with 1 or 2 substituents independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino; and

R¹ and R² are each independently selected from H, halo, CN, NO₂, C₁₋₆ alkyl, C₁₋₆ haloalkyl, C₁₋₆ alkoxy, and C₁₋₆ haloalkoxy.

In some embodiments, W is S, and L¹ and L² are both absent.

In some embodiments, the compound of Formula (I) has formula:

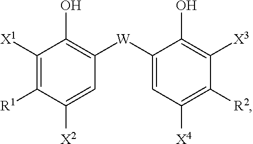

or a pharmaceutically acceptable salt thereof.

In some embodiments, the compound of Formula (I) has formula:

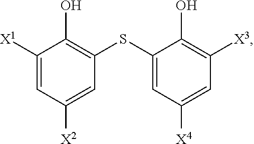

or a pharmaceutically acceptable salt thereof.

In some embodiments, the compound of Formula (I) is selected from any one of the following compounds:

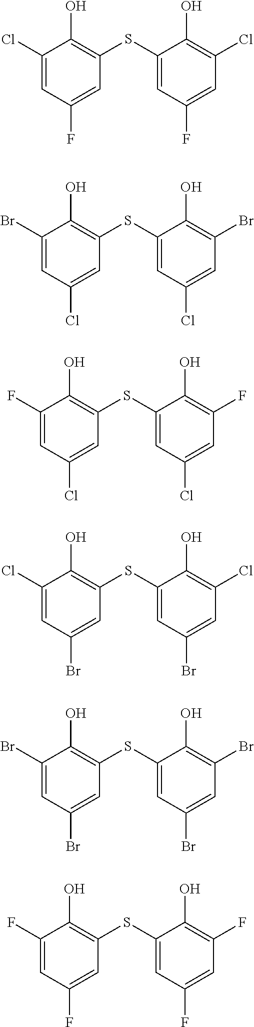

or a pharmaceutically acceptable salt thereof.

In some embodiments, the compound of Formula (I) is selected from any one of the following compounds:

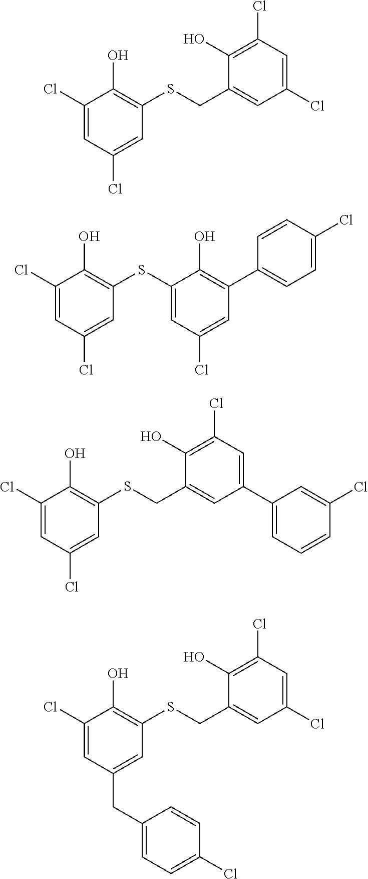

or a pharmaceutically acceptable salt thereof.

In some embodiments, the present disclosure provides a compound of formula (1):

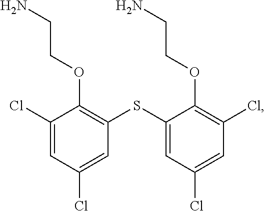 [SMILES: NCCOc1c(Cl)cc(Cl)cc1Sc1cc(Cl)cc(Cl)c1OCCN]

or a pharmaceutically acceptable salt thereof.

In some embodiments, a salt (e.g., pharmaceutically acceptable salt) of a compound of Formula (I) or formula (1) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of Formula (I or formula (1)) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, P-hydroxybutyrate, glycolate, maleate, tartrate, methanesulfonate, propanesulfonate, naphthalene-1-sulfonate, naphthalene-2- sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (I) or formula (1) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C1-C6)-alkylamine), such as N,N-dimethyl-N-(2-hydroxyethyl)amine or tri-(2-hydroxyethyl)amine; N-methyl-D-glucamine; morpholine; thiomorpholine;

piperidine; pyrrolidine; and amino acids such as arginine, lysine, and the like. In some embodiments, the compounds of Formula (I) or formula (1), or pharmaceutically acceptable salts thereof, are substantially isolated.

Methods of Making of Antibacterial Compounds

Compounds of Formula (I) or formula (1), including salts thereof, can be prepared using known organic synthesis techniques and can be synthesized according to any of numerous possible synthetic routes. In some cases, compounds as provided herein are commercially available.

It will be appreciated by one skilled in the art that the processes described are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

In one example, compounds of Formula (I) or formula (1) can be synthesized according to the numerous methods and procedures available to one of ordinary skill in the art. Such methods and procedures can be found, for example, in Smith et al., March's Advanced Organic Chemistry: Reactions, Mechanisms, and Structure, 6th Ed. (Wiley, 2007). Suitable starting materials and intermediates are readily available from various commercial sources. The compounds of Formula (I) or formula (1) may also be prepared using methods analogous to those described in Examples 1 and 9, and shown in FIGS. 5 and 17.

Methods of use

Inhibition of bacterial pathogens

In some embodiments, the present application is directed to a method of killing or inhibiting growth of bacteria, the method comprising contacting the bacteria with an effective amount of a compound of Formula (I) or formula (1), or a pharmaceutically acceptable salt thereof, as described herein. The bacteria may be contacted in vitro, in vivo, or ex vivo. In some embodiments, the compound kills the bacteria by disrupting the bacterial membrane (e.g., making the membrane both permeable and fluid).

In some embodiments, the bacteria (e.g., any one of bacteria described herein) is resistant (or tolerant) to one or more of other antibiotic agents (e.g., antibiotic agents disclosed herein). In some embodiments, the bacteria is at least 2-fold, 4-fold, 8-fold, 10-fold, 24-fold, 48-fold, 100-fold, 256-fold, 512-fold or 1000-fold resistant to one or more of other antibiotic agents. In some embodiments, the bacteria is multi-drug resistant (MDR). In some embodiments, any one of bacteria described herein is resistant to at least one of methicillin, vancomycin, rifampicin, linezolid, daptomycin, gentamicin and ciprofloxacin.

In some embodiments, the bacteria is not resistant to a compound of Formula (I) or formula (1). In some embodiments, the bacteria is at most 1.5-fold or 2-fold resistant to a compound of Formula (I) or formula (1). In some embodiments, any one of bacteria described herein is resistant to one or more of other antibiotic agents and is not resistant to a compound of Formula (I) or formula (1).

In some embodiments, the bacteria is a Gram-positive bacteria. In some embodiments, the present disclosure provides a method of killing or inhibiting growth of a Gram-positive bacteria, the method comprising contacting the bacteria with an effective amount of a compound of Formula (I) or formula (1), or a pharmaceutically acceptable salt thereof. In some embodiments, the present disclosure provides a method of killing or inhibiting growth of a Gram-positive bacteria which is resistant to one or more other antibiotic agents, the method comprising contacting the bacteria with an effective amount of a compound of Formula (I) or formula (1), or a pharmaceutically acceptable salt thereof.

In some embodiments, the bacteria is a member of a genus selected from the group consisting of Staphylococcus (including coagulase negative and coagulase positive), Streptococcus, Peptococcus, Enterococcus, and Bacillus.

In some embodiments, the bacteria is a member of the Staphylococcus genus and the species of bacteria is selected from the group consisting of S. aureus, methicillin-susceptible S. aureus (MSSA), coagulase negative staphylococci, methicillin-resistant S. aureus (MRSA), vancomycin-resistant S. aureus (TZRSA), S. arlettae, S. agnetis, S. auricularis, S. capitis, S. caprae, S. carnosus, S. caseolyticus, S. chromogenes, S. cohnii, S. condimenti, S. delphini, S. devriesei, S. epidermidis, S. equorum, S. felis, S. fleurettii, S. gallinarum, S. haemolyticus, S. hominis, S. hyicus, S. intermedius, S. kloosii, S. leei, S. lentus, S. lugdunensis, S. lutrae, S. massiliensis, S. microti, S. muscae, S. nepalensis, S. pasteuri, S. pettenkoferi, S. piscifermentans, S. pseudintermedius, S. pseudolugdunensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus.

In some embodiments, the bacteria is a member of the Peptococcus genus and the species of bacteria is P. magnus.

In some embodiments, the bacteria is a member of the Streptococcus genus and the species of bacteria is selected from the group consisting of S. agalactiae, S. anginosus, S. bovis, S. canis, S. constellatus, S. dysgalactiae, S. equinus, S. iniae, S. intermedius, S. milleri, S. mitis, S. mutans, S. oxalis, S. parasanguinis, S. peroris, S. pneumoniae, S. pseudopneumoniae, S. pyogenes, S. ratti, S. salivarius, S. tigurinus, S. thermophilus, S. sanguinis, S. sobrinus, S. suis, S. uberis, S. vestibularis, S. viridans, and S. zooepidemicus.

In some embodiments, the bacteria is a member of the Enterococcus genus and the species of bacteria is selected from the group consisting of E. avium, E. durans, E. faecalis, E. gallinarum, E. haemoperoxidus, E. hirae, E. malodoratus, E. moraviensis, E. mundtii, E. pseudoavium, E. raffinosus, E. solitaries, and E. faecium.

In some embodiments, the bacteria is a member of Propionibacterium genus. In such embodiments, the bacteria is P. acnes.

In some embodiments, the bacteria is selected from S. aureus, methicillin-resistant S. aureus (MRSA), vancomycin-resistant S. aureus (VRSA), vancomycin-resistant Enterococcus (VRE), E. faecalis, E. faecium, B. subtilis, and B. anthracis.

In some embodiments, the bacteria is a member of a genus selected from the group consisting of Staphylococcus, Streptococcus, Peptococcus, Enterococcus, and Bacillus.

In some embodiments, the bacteria is a member of a genus selected from the group consisting of Staphylococcus, Enterococcus, and Bacillus.

In some embodiments, the bacteria is a member of a genus selected from the group consisting of Staphylococcus, Enterococcus, Enterobacter, Klebsiella, Pseudomonas, and Acinetobacter.

In some embodiments, the bacteria is a member of a species selected from the group consisting of S. aureus, methicillin-resistant S. aureus (MRSA), vancomycin-resistant S. aureus (VRSA), vancomycin-resistant Enterococcus (VRE), E. spp., K. pneumoniae, P. aeruginosa, A. baumannii, E. faecium, and E. faecalis.

In some embodiments, the bacteria is a Gram-negative bacteria. In some embodiments, the present disclosure provides a method of killing or inhibiting growth of a Gram-negative bacteria, the method comprising contacting the bacteria with an effective amount of a compound of Formula (I) or formula (1), or a pharmaceutically acceptable salt thereof. In some embodiments, the present disclosure provides a method of killing or inhibiting growth of a Gram-negative bacteria which is resistant to one or more other antibiotic agents, the method comprising contacting the bacteria with an effective amount of a compound of Formula (I) or formula (1), or a pharmaceutically acceptable salt thereof.

In some embodiments, the bacteria is a member of a family selected from the group consisting of Enterobacteriaceae, Helicobacteraceae, Campylobacteraceae, Neisseriaceae, Pseudomonadaceae, Moraxellaceae, Xanthomonadaceae, Pasteurellaceae, and Legionellaceae.

In some embodiments, the bacteria is a member of a genus selected from the group consisting of Citrobacter, Enterobacter, Escherichia, Klebsiella, Pantoea, Proteus, Salmonella, Serratia, Shigella, Yersinia, Helicobacter, Wolinella, Campylobacter, Arcobacter, Neisseria, Francisella, Pseudomonas, Acinetobacter, Moraxella, Stenotrophomonas, Haemophilus, Pasteurella, and Legionella.

In some embodiments, the bacteria is a member of the Citrobacter genus and the species of bacteria is selected from the group consisting of C. amalonaticus, C. braakii, C. diversus, C. farmer, C. freundii, C. gillenii, C. koseri, C. murliniae, C. rodentium, C. sedlakii, C. werkmanii, and C. youngae.

In some embodiments, the bacteria is a member of the Enterobacter genus and the species of bacteria is selected from the group consisting of E. aerogenes, E. amnigenus, E. agglomerans, E. arachidis, E. asburiae, E. cancerogenous, E. cloacae, E. cowanii, E. dissolvens, E. gergoviae, E. helveticus, E. hormaechei, E. intermedius, E. kobei, E. ludwigii, E. mori, E. nimpressuralis, E. oryzae, E. pulveris, E. pyrinus, E. radicincitans, E. taylorae, E. turicensis, E. sakazakii, and E. spp.

In some embodiments, the bacteria is a member of the Escherichia genus and the species of bacteria is selected from the group consisting of E. albertii, E. blattae, E. coli, E. fergusonii, E. hermannii, and E. vulneris.

In some embodiments, the bacteria is a member of the Klebsiella genus and the species of bacteria is selected from the group consisting of K. granulomatis, K oxytoca, K. pneumoniae, K terrigena, and K. planticola.

In some embodiments, the bacteria is a member of the Pantoea genus and the species of bacteria is selected from the group consisting of P. agglomerans, P. ananatis, P. citrea, P. dispersa, P. punctata, P. stewartii, P. terrea, and P. vagans.

In some embodiments, the bacteria is a member of the Proteus genus and the species of bacteria is selected from the group consisting of P. hauseri, P. mirabilis, P. myxofaciens, P. penneri, and P. vulgaris.

In some embodiments, the bacteria is a member of the Salmonella genus and the species of bacteria is selected from the group consisting of S. bongori, and S. enterica.

In some embodiments, the bacteria is a member of the Serratia genus and the species of bacteria is selected from the group consisting of S. entomophila, S. ficaria, S. fonticola, S. grimesii, S. liquefaciens, S. marcescens, S. odorifera, S. plymuthica, S. proteamaculans, S. quinivorans, S. rubidaea, and S. symbiotica.

In some embodiments, the bacteria is a member of the Shigella genus and the species of bacteria is selected from the group consisting of S. boydii, S. dysenteriae, S. flexneri, and S. sonnei.

In some embodiments, the bacteria is a member of the Yersinia genus and the species of bacteria is selected from the group consisting of Y. pestis, Y. pseudotuberculosis, and Y. enterocolitica.

In some embodiments, the bacteria is a member of the Helicobacter genus and the species of bacteria is selected from the group consisting of H. acinonychis, H. anseris, H. aurati, H. baculiformis, H. bilis, H. bizzozeronii, H. brantae, H. canadensis, H. canis, H. cetorum, H. cholecystus, H. cinaedi, H. cynogastricus, H. equorum, H. felis, H. fennelliae, H. ganmani, H. heilmannii, H. hepaticus, H. mesocricetorum, H. macacae, H. marmotae, H. mastomyrinus, H. mesocricetorum, H. muridarum, H. mustelae, H. pametensis, H. pullorum, H. pylori, H. rappini, H. rodentium, H. salomonis, H. suis, H. trogontum, H. typhlonius, and H. winghamensis.

In some embodiments, the bacteria is a member of the Campylobacter genus and the species of bacteria is selected from the group consisting of C. avium, C. butzleri, C. canadensis, C. cinaedi, C. coli, C. concisus, C. corcagiensis, C. cryaerophilus, C. cuniculorum, C. curvus, C. fennelliae, C. fetus, C. gracilis, C. helveticus, C. hominis, C. hyoilei, C. hyointestinalis, C. insulaenigrae, C. jejuni, C. lanienae, C. lari, C. mucosalis, C. mustelae, C. nitrofigilis, C. peloridis, C. pylori, C. rectus, C. showae, C. sputorum, C. subantarcticus, C. upsaliensis, C. ureolyticus, and C. volucris.

In some embodiments, the bacteria is a member of the Arcobacter genus and the species of bacteria is selected from the group consisting of A. bivalviorum, A. butzleri, A. cibarius, A. cryaerophilus, A. defluvii, A. ellisii, A. halophilus, A. marinus, A. molluscorum, A. mytili, A. nitrofigilis, A. skirrowii, A. thereius, A. trophiarum, and A. venerupis.

In some embodiments, the bacteria is a member of the Neisseria genus and the species of bacteria is selected from the group consisting of N. bacilliformis, N. cinerea, N. denitrificans, N. elongata, N. flavescens, N. gonorrhoeae, N. lactamica, N. macacae, N. meningitidis, N. mucosa, N. pharyngis, N. polysaccharea, N. sicca, N. subflava, and N. weaver.

In some embodiments, the bacteria is a member of the Francisella genus and the species of bacteria is selected from the group consisting of F. tularensis, F. novicida, F. hispaniensis, W. persica, F. noatunensis, F. philomiragia, F. halioticida, F. endociliophora, and F. guangzhouensis.

In some embodiments, the bacteria is a member of the Pseudomonas genus and the species of bacteria is selected from the group consisting of P. aeruginosa, P. oryzihabitans, and P. plecoglossicida.

In some embodiments, the bacteria is a member of the Acinetobacter genus and the species of bacteria is A. baumannii.

In some embodiments, the bacteria is a member of the Moraxella genus and the species of bacteria is selected from the group consisting of M catarrhalis, M lacunata, and M bovis.

In some embodiments, the bacteria is a member of the Stenotrophomonas genus and the species of bacteria is S. maltophilia.

In some embodiments, the bacteria is a member of the Haemophilus genus and the species of bacteria is selected from the group consisting of H. aegyptius, H. aphrophilus, H. avium, H. ducreyi, H. felis, H. haemolyticus, H. influenzae, H. parainfluenzae, H. paracuniculus, H. parahaemolyticus, H. pittmaniae, Haemophilus segnis, and H. somnus.

In some embodiments, the bacteria is a member of the Pasteurella genus and the species of bacteria is selected from the group consisting of P. multocida, P. stomatis, P. dagmatis, P. canis, P. bettyae, and P. anatis.

In some embodiments, the bacteria is a member of the Legionella genus and the species of bacteria is selected from the group consisting of L. pneumophila, L. anisa, L. bozemanae, L. cincinnatiensis, L. gormanii, L. jordani, L. longbeachae, L. maceachernii, L. micdadei, L. sainthelensi, L. wadsworthii, and L. waltersii.

In some embodiments, the bacteria is a member of the Mycobacterium genus and the species of bacteria is selected from a group consisting of M. tuberculosis and M. smegmatic.

In some embodiments, the bacteria is a member of a genus selected from: Acinetobacter, Burkholderia, Acinetobacter, Burkholderia, Klebsiella, Pseudomonas, and Escherichia. In such embodiments, the bacteria is a member of a species selected from: K pneumoniae, P. aeruginosa, Enterobacteriaceae, and E. coli.

In some embodiments, the present disclosure provides a method of killing or inhibiting growth of bacteria (e.g., any bacteria described herein), the method comprising the steps of:

(i) determining that the bacteria is tolerant or resistant (e.g., least 2-fold, at least 4-fold, at least 8-fold, at least 10-fold, at least 24-fold, at least 48-fold, at least 100-fold, at least 256-fold, at least 512-fold, or at least 1000-fold resistant) to one or more other antibiotic agents (e.g., any one of antibiotic agents described herein); and

(ii) contacting the bacteria with an effective amount of a compound of Formula (I) or formula (1) described herein.

Treating bacterial infections

In some embodiments, the present disclosure provides a method of treating (or preventing) a bacterial infection in a subject, the method comprising administering to the subject in need thereof a therapeutically effective amount of a compound of

Formula (I) or formula (1), or a pharmaceutically acceptable salt thereof, as described herein. In some embodiments, the present disclosure provides a method of treating (or preventing) a bacterial infection caused by Gram-positive bacteria, the method comprising administering to the subject in need thereof a therapeutically effective amount of a compound of Formula (I) or formula (1), or a pharmaceutically acceptable salt thereof. In some embodiments, the present disclosure provides a method of treating (or preventing) a bacterial infection caused by Gram-positive bacteria which is tolerant or resistant to one or more other antibiotic agents, the method comprising administering to the subject in need thereof a therapeutically effective amount of a compound of Formula (I) or formula (1), or a pharmaceutically acceptable salt thereof.

In some embodiments, the bacterial infection is caused by any one of the bacteria described herein (e.g., MRSA). In some embodiments, the bacterial infection is resistant (or tolerant) to treatment with one or more of the antibiotic agents described herein (e.g., bacterial infection is resistant to treatment with methicillin, vancomycin, rifampicin, gentamicin and/or ciprofloxacin). In these embodiments, the bacterial infection is characterized as resistant (or tolerant) to treatment with one or more available antibiotic agents.

In some embodiments, the bacterial infection is selected from the group consisting of nosocomial infection, skin infection, respiratory infection, wound infection, endovascular infection, CNS infection, abdominal infection, blood stream infection, urinary tract infection, pelvic infection, invasive systemic infection, gastrointestinal infection, dental infection, zoonotic infection, and connective tissue infection.

In some embodiments, the bacterial infection is a skin infection. In some aspects of these embodiments, the skin infection is selected from the group consisting of acne, pimples, impetigo, boils, cellulitis, folliculitis, carbuncles, scalded skin syndrome, skin abscesses, atopic dermatitis, and typhoid fever.

In some embodiments, the bacterial infection is a respiratory infection. In some aspects of these embodiments, the respiratory infection is selected from the group consisting of upper respiratory tract infection, bronchopneumonia, atypical pneumonia, tuberculosis, mycobacterium tuberculosis, pneumonia, anaerobic pleuropulmonary infection, ventilator-associated pneumonia, aspiration pneumonia, lung abscess, bronchitis, chronic obstructive pulmonary disease, obstructive pulmonary disease, Pontiac fever, and legionellosis.

In some embodiments, the bacterial infection is a wound infection. In some aspects of these embodiments, the wound infection is a postsurgical wound infection. In some embodiments, the bacterial infection is a blood stream infection. In some aspects of these embodiments, the blood stream infection is bacteremia or sepsis. In some embodiments, the bacterial infection is a pelvic infection. In some aspects of the embodiments, the pelvic infection is bacterial vaginosis.

In some embodiments, the bacterial infection is a gastrointestinal infection. In some aspects of these embodiments, the gastrointestinal infection is selected from the group consisting of peptic ulcer, chronic gastritis, duodenitis, gastroenteritis, diarrhea, dysentery, diphtheria, food poisoning and foodborne illness.

In some embodiments, the bacterial infection is a bone, joint or muscle infection. In some aspects of these embodiments, the bone, joint or muscle infection is selected from the group consisting of tetanus, secondary meningitis, meningitis, neonatal meningitis, sinusitis, laryngitis, arthritis, septic arthritis, Bartholin gland abscess, chancroid, osteomyelitis, endocarditis, mediastinitis, pericarditis, peritonitis, otitis media, blepharoconjunctivitis, keratoconjunctivitis, and conjunctivitis.

In some embodiments, the bacterial infection is selected from the group consisting of a dental infection, a zoonotic infection, an invasive systemic infection, a urinary tract infection, an abdominal infection, a CNS infection, an endovascular infection, and a nosocomial infection. In some embodiments, the bacterial infection is selected from the group consisting of syphilis, leprosy, abscesses, sepsis, empyema, and tularemia.

In some embodiments, the bacterial infection is associated with implanted devices (e.g., catheter, ballon catheter, stent, pacer etc). In some embodiments, the bacterial infection is osteomyelitis, endocarditis, or an infection associated with an implanted device, which is caused by a S. aureus persister.

In some embodiments, the bacterial infection is a connective tissue infection, or a joint or muscle infection. In such embodiments, the joint infection is an infection of a shoulder, a knee, a hip, or an elbow. In some embodiments, the bacterial infection is septic arthritis (e.g., septic arthritis caused by S. aureus). In some embodiments, the infection is selected from atopic dermatitis, sinusitis, food poisoning, abscess, pneumonia, meningitis, osteomyelitis, endocarditis, bacteremia, sepsis, and urinary tract infection.

Combination Treatments

Is some embodiments, the present disclosure provides a method of treating (or preventing) a bacterial infection in a subject, the method comprising administering to the subject in need there of a therapeutically effective amount of a compound of Formula (I) or formula (1), or a pharmaceutically acceptable salt thereof, in combination with at least one additional therapeutic agent, or a pharmaceutically acceptable salt thereof.

In some embodiments, a method of treating a bacterial infection in a subject in need thereof (as described herein) comprises administering to the subject one or more additional therapeutic agents, or a pharmaceutically acceptable salt thereof.

In some embodiments, a pharmaceutical composition of the present application comprises a compound of Formula (I) or formula (1), or a pharmaceutically acceptable salt thereof, and at least one additional therapeutic agent, or a pharmaceutically acceptable salt thereof.

The additional therapeutic agent may be selected from any compound or therapeutic agent known to have or that demonstrates advantageous properties when administered with a compound of Formula (I) or formula (1), or a pharmaceutically acceptable salt thereof. In some embodiments, the additional therapeutic agent is any one of the antibiotics described herein (e.g., gentamicin, daptomycin, or nTZDpa).

The additional therapeutic agent may be administered to the subject in a therapeutically effective amount. An effective amount of the additional therapeutic agent is typically between about 20% and 100% of the dosage normally utilized in a monotherapy regime using just that therapeutic agent. In one example, an effective amount is between about 70% and 100% of the normal monotherapeutic dose. The normal monotherapeutic dosages of these additional therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif (2000), each of which references are incorporated herein by reference in their entirety. In some embodiments, when the additional therapeutic agent is gentamicin, the effective amount of gentamicin is lower than the amount that causes nephrotoxicity in a subject. The additional therapeutic agent may be administered to the subject in a separate pharmaceutical composition or dosage form (e.g., any one of the compositions, formulation, routes and dosage forms described herein).

In some embodiments, the additional therapeutic agent is an antibiotic.

In some embodiments, the antibiotic is a cationic antimicrobial peptide (CAMP). In some aspects of these embodiments, the cationic antimicrobial peptide is a defensin peptide (e.g., defensin 1 such as beta-defensin 1 or alpha-defensin 1), or cecropin, andropin, moricin, ceratotoxin, melittin, magainin, dermaseptin, bombinin, brevinin (e.g., brevinin-1), esculentin, buforin II (e.g., from amphibians), CAP18 (e.g., from rabbits), LL37 (e.g., from humans), abaecin, apidaecins (e.g., from honeybees), prophenin (e.g., from pigs), indolicidin (e.g., from cattle), brevinins, protegrin (e.g., from pig), tachyplesins (e.g., from horseshoe crabs), or drosomycin (e.g., from fruit flies).

In some embodiments, the antibiotic is selected from the quinolone class of antibiotic compounds. In some aspects of these embodiments, the antibiotic is selected from the group consisting of levofloxacin, norfloxacin, ofloxacin, ciprofloxacin, perfloxacin, lomefloxacin, fleroxacin, sparfloxacin, grepafloxacin, trovafloxacin, clinafloxacin, gemifloxacin, enoxacin, sitafloxacin, nadifloxacin, tosulfloxacin, cinnoxacin, rosoxacin, miloxacin, moxifloxacin, gatifloxacin, cinnoxacin, enoxacin, fleroxacin, lomafloxacin, lomefloxacin, miloxacin, nalidixic acid, nadifloxacin, oxolinic acid, pefloxacin, pirimidic acid, pipemidic acid, rosoxacin, rufloxacin, temafloxacin, tosufloxacin, trovafloxacin, and besifloxacin.

In some embodiments, the antibiotic is selected from a β-lactam, a monobactam, oxazolidinone, and lipopeptide.

In some embodiments, the antibiotic is selected from the cephalosporin class of antibiotic compounds. In some aspects of these embodiments, the antibiotic is selected from the group consisting of cefazolin, cefuroxime, ceftazidime, cephalexin, cephaloridine, cefamandole, cefsulodin, cefonicid, cefoperazine, cefoprozil, and ceftriaxone.

In some embodiments, the antibiotic is selected from the penicillin class of antibiotic compounds. In some aspects of these embodiments, the antibiotic is selected from the group consisting of penicillin G, penicillin V, procaine penicillin, and benzathine penicillin, ampicillin, and amoxicillin, benzylpenicillin, phenoxymethylpenicillin, oxacillin, methicillin, dicloxacillin, flucloxacillin, temocillin, azlocillin, carbenicillin, ricarcillin, mezlocillin, piperacillin, apalcillin, hetacillin, bacampicillin, sulbenicillin, mecicilam, pevmecillinam, ciclacillin, talapicillin, aspoxicillin, cloxacillin, nafcillin, and pivampicillin.

In some embodiments, the antibiotic is selected from the carbapenem class of antibiotic compounds. In some aspects of these embodiments, the antibiotic is selected from the group consisting of thienamycin, tomopenem, lenapenem, tebipenem, razupenem, imipenem, meropenem, ertapenem, doripenem, panipenem (betamipron), and biapenem.

In some embodiments, the antibiotic is selected from the lipopeptide class of antibiotic compounds. In some aspects of these embodiments, the antibiotic is selected from the group consisting of polymyxin B, colistin (polymyxin E), and daptomycin.

In some embodiments, the antibiotic is selected from the aminoglycoside class of antibiotic compounds. In some aspects of these embodiments, the antibiotic is selected from the group consisting of gentamicin, amikacin, tobramycin, debekacin, kanamycin, neomycin, netilmicin, paromomycin, sisomycin, spectinomycin, and streptomycin.

In some embodiments, the antibiotic is selected from the glycopeptide class of antibiotic compounds. In some aspects of these embodiments, the antibiotic is selected from the group consisting of vancomycin, teicoplanin, telavancin, ramoplanin, daptomycin, decaplanin, and bleomycin.

In some embodiments, the antibiotic is selected from the macrolide class of antibiotic compounds. In some aspects of these embodiments, the antibiotic is selected from the group consisting of azithromycin, clarithromycin, erythromycin, fidaxomicin, telithromycin, carbomycin A, josamycin, kitasamycin, midecamycin/midecamycinacetate, oleandomycin, solithromycin, spiramycin, troleandomycin, tylosin/tylocine, roxithromycin, dirithromycin, troleandomycin, spectinomycin, methymycin, neomethymycin, erythronolid, megalomycin, picromycin, narbomycin, oleandomycin, triacetyl-oleandomycin, laukamycin, kujimycin A, albocyclin and cineromycin B.

In some embodiments, the antibiotic is selected from the ansamycin class of antibiotic compounds. In some aspects of these embodiments, the antibiotic is selected from the group consisting of streptovaricin, geldanamycin, herbimycin, rifamycin, rifampin, rifabutin, rifapentine and rifamixin.

In some embodiments, the antibiotic is selected from the sulfonamide class of antibiotic compounds. In some aspects of these embodiments, the antibiotic is selected from the group consisting of sulfanilamide, sulfacetarnide, sulfapyridine, sulfathiazole, sulfadiazine, sulfamerazine, sulfadimidine, sulfasomidine, sulfasalazine, mafenide, sulfamethoxazole, sulfamethoxypyridazine, sulfadimethoxine, sulfasymazine, sulfadoxine, sulfametopyrazine, sulfaguanidine, succinylsulfathiazole and phthalylsulfathiazole.

In some embodiments, the antibiotic is selected from the group consisting of quinolones, fluoroquinolones, β-lactams, cephalosporins, penicillins, carbapenems, lipopeptide antibiotics, glycopeptides, macrolides, ansamycins, sulfonamides, and combinations of two or more thereof.

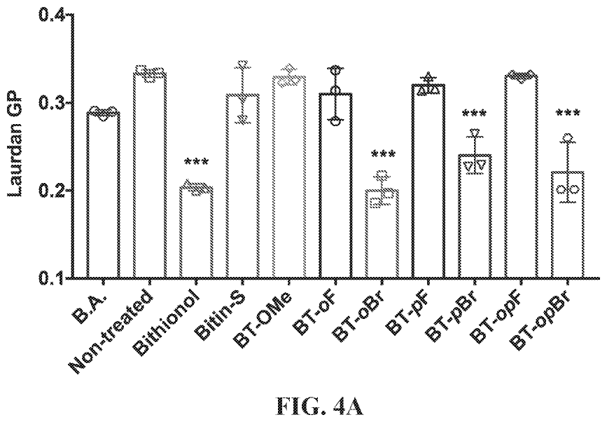

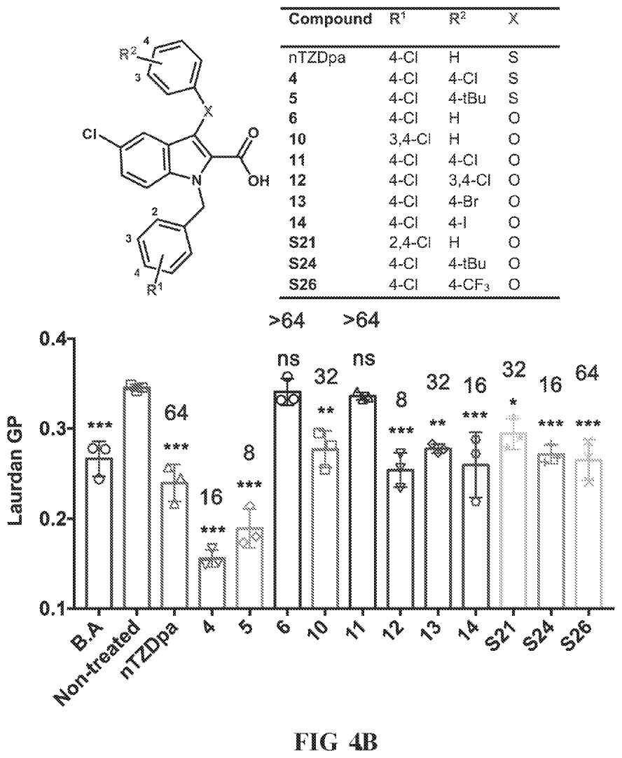

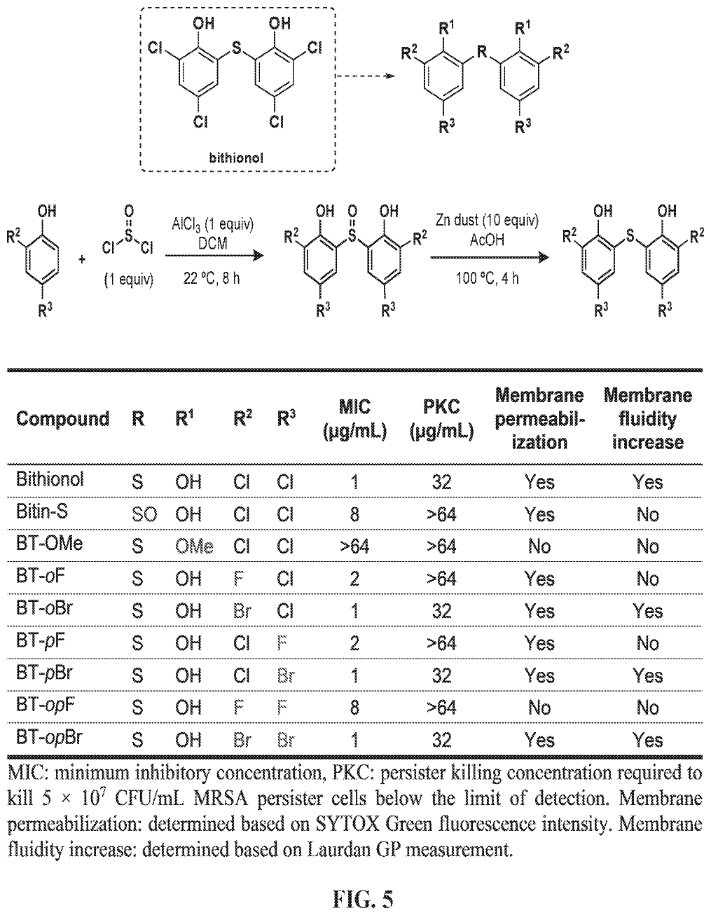

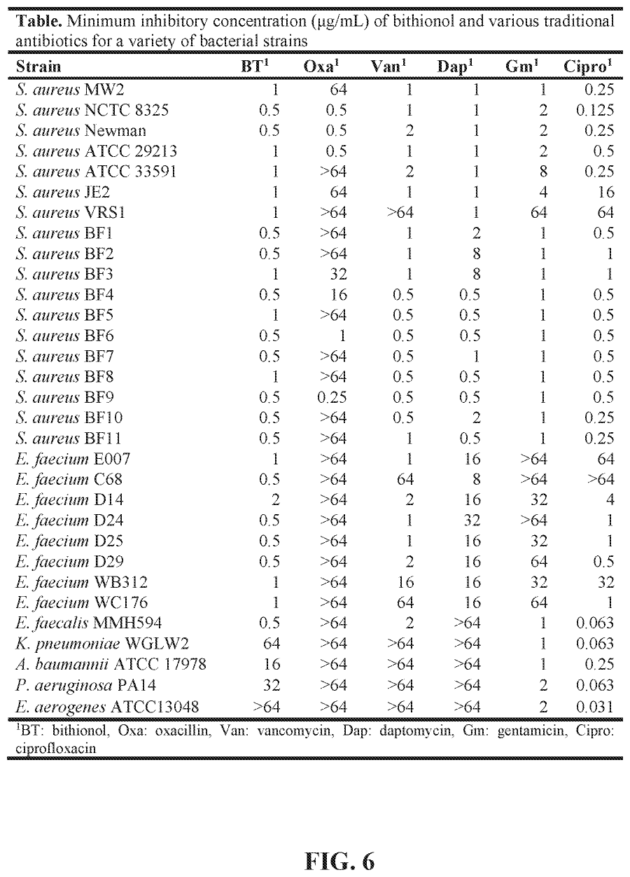

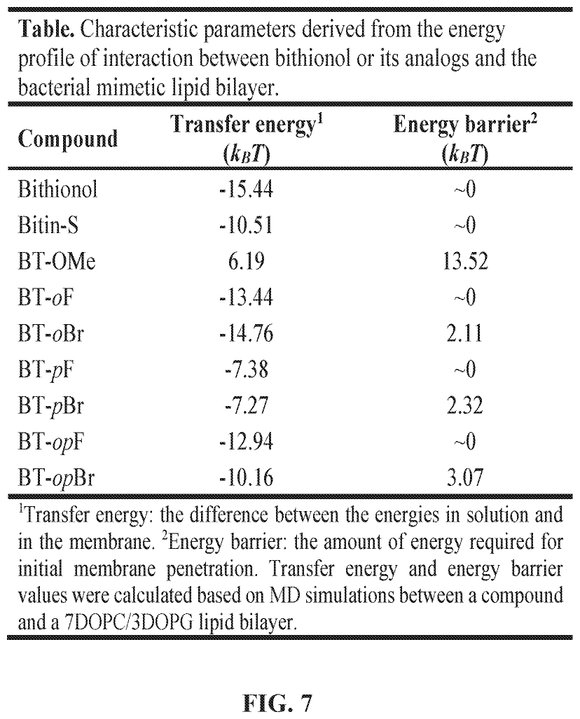

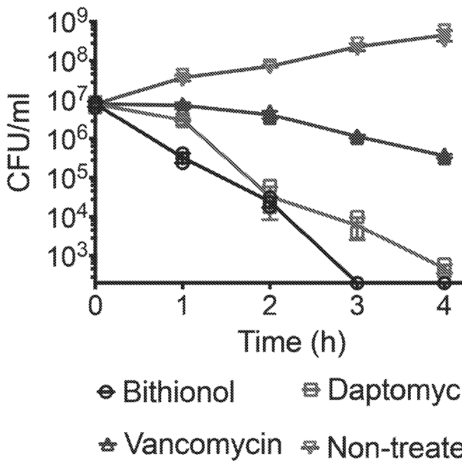

In some embodiments, the antibiotic is a membrane-active or membrane-disrupting agent. Suitable examples of such compounds are shown in FIG. 4B, and are described in WO 2019/199979, which is incorporated herein by reference in its entirety. Other examples of membrane-active antibiotic agents are described, for example, in WO 2018/213609 and WO 2017/053778, which are incorporated hereby by reference in their entirety. In some embodiments, the antibiotic is nTZDpa, or a pharmaceutically acceptable salt thereof. In some embodiments, the additional therapeutic agent is selected from gentamicin and nTZDpa, or a pharmaceutically acceptable salt thereof. In some embodiments, the additional therapeutic agent is daptomycin, or a pharmaceutically acceptable salt thereof.

In some embodiments, the present application provides separate dosage forms of a compound of Formula (I) or formula (1), or a pharmaceutically acceptable salt thereof, and one or more of any of the above-described second therapeutic agents. The separate dosage forms may be administered together consecutively (e.g., within less than 24 hours of one another) or simultaneously (e.g., administered to the patient within 5 minutes of one another).

Some of the second therapeutic agents referenced above will act synergistically with the compounds of the present application. In some embodiments, some of the second therapeutic agents referenced above will show additive effect. When this occurs, it will allow the effective dosage of the second therapeutic agent and/or the compound of the present application to be reduced from that required in a monotherapy. Preferably, the combination will have an advantage of minimizing toxic side effects of either the second therapeutic agent of a compound of the present application, synergistic improvements in efficacy, improved ease of administration or use and/or reduced overall expense of compound preparation or formulation.

Compositions, Formulations, and Routes of Administration

In some embodiments, the present application also provides pharmaceutical compositions comprising an effective amount of a compound of Formula (I) or formula (1), or a pharmaceutically acceptable salt thereof; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

If required, the solubility and bioavailability of the compounds of the present application in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of the present application optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the present application include those suitable for oral, rectal, nasal, topical (including buccal and sublingual), vaginal or parenteral (including subcutaneous, intramuscular, intravenous and intradermal) administration. In certain embodiments, the compound of Formula (I) or formula (1) herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product. In some embodiments, the compound of Formula (I) or formula (1), or a pharmaceutically acceptable salt thereof, is administered orally. Compositions of the present application suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031.

Topical administration of the pharmaceutical compositions of the present application is especially useful when the desired treatment involves areas or organs readily accessible by topical application (e.g., skin and soft tissues).

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form, as a cream or a paste.

In some embodiments, the topical composition comprises a combination of a compound of Formula (I) or formula (1), or a pharmaceutically acceptable salt thereof, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Application of the subject therapeutics may be local, so as to be administered at the site of interest (e.g., infected area of skin, or an infected joint or other connective tissue). Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides a method of coating an implantable medical device comprising the step of contacting said device with the coating composition described above. It will be obvious to those skilled in the art that the coating of the device will occur prior to implantation into a mammal.

According to another embodiment, the present application provides a method of impregnating an implantable drug release device comprising the step of contacting said drug release device with a compound or composition of the present application. Implantable drug release devices include, but are not limited to, biodegradable polymer capsules or bullets, non-degradable, diffusible polymer capsules and biodegradable polymer wafers.

According to another embodiment, the present application provides an implantable medical device coated with a compound or a composition comprising a compound of the present application, such that said compound is therapeutically active.

Where an organ or tissue is accessible because of removal from the subject, such organ or tissue may be bathed in a medium containing a composition of the present application, a composition of the present application may be painted onto the organ, or a composition of the present application may be applied in any other convenient way.

In the pharmaceutical compositions of the present application, a compound of any one of Formula (I) or formula (1), or a pharmaceutically available salt thereof, is present in an effective amount (e.g., a therapeutically effective amount).

The interrelationship of dosages for animals and humans (based on milligrams per meter squared of body surface) is described in Freireich et al., Cancer Chemother.

Rep, 1966, 50: 219. Body surface area may be approximately determined from height and weight of the subject. See, e.g., Scientific Tables, Geigy Pharmaceuticals, Ardsley, N.Y., 1970, 537.

In some embodiments, an effective amount of a compound of Formula (I) or formula (1), or a pharmaceutically acceptable salt thereof, can range, for example, from about 1mg to about 200 mg, from about 1 to about 100 mg, from about 1 to about 50 mg, from about 1 mg to about 30 mg, from about 1 mg to about 15 mg, from about 10 mg to about 2000 mg, from about 10 mg to about 1900 mg, from about 10 mg to about 1800 mg, from about 10 mg to about 1700 mg, from about 10 mg to about 1600 mg, from about 10 mg to about 1500 mg, from about 10 mg to about 1400 mg, from about 10 mg to about 1300 mg, from about 10 mg to about 1200 mg, from about 10 mg to about 1100 mg, from about 10 mg to about 1000 mg, from 10 mg about to about 900 mg, from about 10 mg to about 800 mg, from about 10 mg to about 700 mg, from about 10 mg to about 600 mg, from about 10 mg to about 500 mg, from about 10 mg to about 400 mg, from about 10 mg to about 300 mg, from about 10 mg to about 200 mg, from about 10 mg to about 100 mg, and from about 10 mg to about 50 mg. In some embodiments, an effective amount of a compound of Formula (I) or formula (1), or a pharmaceutically acceptable salt thereof, is 1 mg, 2 mg, 3 mg, 4 mg, 5 mg, 6 mg, 7 mg, 8 mg, 9 mg, or 10 mg. In some aspects of these embodiments, the composition containing an effective amount of a compound of Formula (I) or formula (1), or a pharmaceutically acceptable salt thereof, is administered once daily. In some aspects of these embodiments, the composition containing an effective amount of a compound of Formula (I) or formula (1), or a pharmaceutically acceptable salt thereof, is administered twice daily. In some aspects of these embodiments, the composition containing an effective amount of a compound of Formula (I) or formula (1), or a pharmaceutically acceptable salt thereof, is administered thrice daily.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

Cleaning Compositions

In some embodiments, any one of compounds of Formula (I) or formula (1), or a salt thereof, may be used for killing bacteria on a surface (e.g., for disinfecting or sanitizing a surface). The surface may be metallic, plastic, ceramic, or wooden, for example, the surface is a floor, a table, a kitchen counter, a cutting board, or a medical instrument. Hence, any one of the compounds of the present application may be used in a commercial setting for general disinfecting, e.g., in medical and food industries. For these purposes, the compound may be provided in a cleaning composition comprising an acceptable carrier. The carrier(s) are “acceptable” in the sense of being compatible with the other ingredients of the cleaning composition. Acceptable carriers that may be used in a cleaning composition of the present application include, but are not limited to, alcohols, water, surfactants, emollients, stabilizers, thickeners, viscosifiers, and fragrances.

Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cn_m” indicates a range which includes the endpoints, wherein n and m are integers and indicate the number of carbons. Examples include C_{1-4,} C_{1-6,} and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tent-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tent-butoxycarbonyl), and the like.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tent-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(C. alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)-group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), 5(O), C(S), or S(O)_{2,} etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C=0), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H- 1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system, an in vivo system, or an ex vivo system. For example, “contacting” the bacteria with a compound of the invention includes the administration of a compound of the present invention to an individual or patient, such as a human, having the bacteria, as well as, for example, introducing a compound of the invention into a sample containing a bacteria.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “resistant” refers to bacteria that has acquired genetic resistance to antibiotics. In one example, MRSA is typically resistant to multiple antibiotic agents at the same time due to the presence of mecA gene in the staphylococcal cassette chromosome mec (SCCmec) of various types.

As used herein, the terms “persistent” and “persister” refer to bacteria that has acquired transitory tolerance, as opposed to genetic resistance, to antibiotics due to an epigenetic processes that produce antibiotic-tolerant cells that can revert to antibiotic-susceptible cells at a relatively high frequency. For example, in a laboratory setting, the proportion of antibiotic-tolerant (or persistent) cells in an S. aureus population varies depending on growth phase. In a stationary phase, essentially the entire bacterial population is antibiotic-tolerant and will survive prolonged treatment with high concentrations of bactericidal antibiotics. In contrast, in lag and early exponential phase, only a small subpopulation of an S. aureus culture will survive antibiotic treatment due to tolerance to antibiotics. In this latter case, the survivor cells are persisters.

### EXAMPLES

Materials and Methods

Bacterial strains and growth conditions. Bacterial strains used in this study are listed in FIG. 6. S. aureus strains were grown in tryptic soy broth (TSB) (BD, Franklin Lakes, N.J., USA). E. faecium and E. faecalis strains were grown in brain-heart infusion (BHI) broth (BD, Franklin Lakes, N.J., USA), and four Gram-negative species, Klebsiella pneumoniae, Acinetobacter baumannii, Pseudomonas aeruginosa, and Enterobacter aerogenes were grown in Luria Bertani (LB) broth (BD, Franklin Lakes, N.J., USA). All bacteria were grown at 37° C. at 200 r.p.m.

Antimicrobial agents and chemicals. Bithionol, vancomycin, oxacillin, gentamicin, ciprofloxacin, and benzyldimeth:ₛ7lhexa.decyl ammonium chloride (16-BAC) were purchased from Sigma-Aldrich (St Louis, Mo., USA). Bitin-S was purchased from MedChem Express (Monmouth Junction, N.J., USA). nTZDpa, linezolid, and daptomycin were purchased from R&D Systems (Minneapolis, Minn., USA). The synthesis procedure of 7 bithionol analogs are described in Example 1. The synthetic methodology of 11 nTZDpa analogs was previously described in WO 2019/199979, which is incorporated herein by reference in its entirety. All compounds were dissolved in DMSO or ddH₂O to make 10 ing/mL stocks. All assays using daptomycin were supplemented with CaCl₂ at a final concentration of 50 μg/mL.

Minimal inhibitory concentration (MIC) assay. The MICs of antibiotics were determined by the standard micro-dilution method recommended by the Clinical and Laboratory Standards Institute. MIC assays were conducted in biological triplicate.

Killing kinetics assay. An overnight culture of S. aureus MW2 ⁻was diluted in 25 mL TSB to a concentration of ˜10⁵ CFU/mL. The diluted culture was grown to exponential-phase (˜2×10⁷ CFU/mL) at 37° C. with shaking at 200 rpm. 1 mL of the exponential-phase culture was added to each well of a 96-well assay block (Corning Costar 3960) containing 1 mL of 2× of the desired concentrations of antibiotics dissolved in pre-warmed TSB at 37° C. The assay block was then incubated at 37° C. with shaking at 200 rpm. At every hour, 400 μl cultures were removed, washed once with PBS, serially diluted and spot-plated onto tryptic soy agar (TSA, BD) plates. The plates were incubated at 37° C. overnight, and colonies were counted to determine the titer of the cultures. These experiments were conducted in biological triplicate.

Persister killing assay. As has been previously demonstrated, 100% of S. aureus cells in a liquid culture become persisters when grown to stationary phase. Persister cells of MRSA MW2 or VRSA VRS1 were prepared by growing cultures overnight to stationary phase at 37° C. at 200 rpm and washing three times with PBS. 1 mL of ˜1×10⁸ CFU/mL MRSA persisters was added to 1 mL of PBS containing a 2-fold higher concentration of the desired concentration of antibiotics in a 96-well assay block (Corning Costar 3960). For assays with daptomycin, PBS was supplemented with 50 μg/mL CaCl₂. The assay block was incubated at 37° C. with shaking at 200 rpm. At various times, 400 μl samples were removed, washed once with PBS, serially diluted and spot-plated on TSA plates. Colonies were counted after overnight incubation at 37° C. to determine the titer of live cells. These experiments were conducted in triplicate.

Biofilm persister killing assay. An overnight culture of S. aureus MW2 cells was diluted 1:200 with TSB supplemented with 0.2% glucose and 3% NaCl. A 13 mm diameter Millipore mixed cellulose ester membrane (EMD Millipore GSWP01300) was placed at the bottom of each well of a 12-well assay plate (Falcon 353043). 1 of the diluted culture was added to each well and statically incubated at 37° C. for 24 h. The membranes were washed 3-times with PBS and transferred to a fresh 12-well plate, 1 mL PBS containing the desired concentration of antibiotics was added to each well, and then the plate was statically incubated at 37° C. for 24 h, The membranes were washed 3 times with PBS, placed in 2-mL microcentrifuge tubes containing 1 mL PBS, and sonicated in an ultrasonic bath (Fisher Scientific FS 30) for 10 min. The sonicated samples were serially diluted, spot-plated on TSA to enumerate the number of live cells. The experiment was conducted in biological triplicate.

Bacterial membrane permeability assay. Exponential-phase or stationary-phase S. aureus MW2 cells prepared as described above under Killing kinetics assay and Persister killing assay, respectively, were washed 3 times with the same volume of PBS. The washed cells were adjusted to OD₆₀₀=0.4 with PBS. SYTOX Green (Molecular Probes, Waltham, Mass., USA) was added to 10 mL of the diluted bacterial suspension to a final concentration of 5 μM and incubated for 30 min at room temperature in the dark. 50 μl of the bacterial/SYTOX Green suspension was mixed with 50 μl of phosphate buffered saline (PBS) containing twice the desired concentrations of compounds in black, clear-bottom, 96-well plates (Corning no. 3904). Fluorescence was measured at room temperature using a spectrophotometer (SpectraMax M2, Molecular Devices) with excitation and emission wavelengths of 485 nm and 525 nm, respectively. All experiments were conducted in biological triplicate.

Mammalian membrane permeability assay. Human renal proximal tubular epithelial cells (HKC-8) were grown in Dulbecco' s modified Eagle F-12 media mixed 1:1 with Ham's F-12 (DMEM/F-12, Life Technologies, Carlsbad, Calif., USA) supplemented with 10% FBS and 4 mM L-glutamine at 37° C. in 5% CO₂. For membrane permeability assays, HKC-8 was cultured in black, clear-bottom, 96-well plates (Corning no. 3904) in 100 μl/well of the growth media to reach ˜70% confluence. After washing the cells twice with PBS, 50 μl of 0.25 μM SYTOX Green was added to each well. After incubating the cells at 37° C. in 5% CO₂ for 15 min, the cells were treated with a range of concentrations of bithionol or the cholesterol binding detergent saponin (positive control). Fluorescence was measured at room temperature using a spectrophotometer (SpectraMax M2, Molecular Devices) with excitation and emission wavelengths of 485 nm and 525 nm, respectively. The assay was carried out in biological duplicate.

To visualize HKC-8 cells with compromised membranes, HKC-8 was cultured in 24-well plates (Corning no. 3527) until —70% confluence. After washing the cells twice with PBS, 500 μl of 1 μM SYTOX Green was added to each well. The cells were incubated at 37° C. in 5% CO₂ for 15 min and then treated with 32 μg/mL (final concentration) bithionol or saponin for 1 h. Bright field and fluorescent images (Eₓ=470/40 nm, Eₘ=525/40 nm) were obtained using a Zeiss Axiovert 200 microscope with a 10× objective and 10× eyepiece.

Membrane fluidity assay. Bacterial membrane fluidity was estimated by slightly modifying previously described methods. An overnight culture of S. aureus MW2 was diluted 1:1,000 in 25 mL TSB. The diluted culture was grown to OD₆₀₀˜1.5 at 37° C. with shaking at 200 rpm, followed by 10 min incubation with 10 μM Laurdan at room temperature in the dark (Sigma-Aldrich, Cat # 40227). The stained cell culture was washed with PBS 4 times and concentrated 2 times. 100 μL of the 2-fold concentrated culture was mixed with 100 μL of phosphate buffered saline (PBS) containing twice the desired concentrations of compounds in black, clear-bottom, 96-well plates (Corning no. 3904). After 1 h incubation at room temperature in the dark, the Laurdan fluorescence intensities were measured using a spectrophotometer (SpectraMax M2, Molecular Devices) with emission wavelengths of 435 nm and 490 nm upon excitation at 350 nm. The Laurdan GP was calculated using the formula GP=(I₄₃₅−I₄₉₀)/(I₄₃₅+I₄₉₀). All experiments were conducted in biological triplicate.

Human blood hemolysis. Hemolytic activity was evaluated as described in a previous study. Human erythrocytes were purchased from Rockland Immunochemicals (Limerick, Pa., USA). Briefly, 100 μl of human erythrocytes (4% in PBS) was added to 100 μl of two-fold serial dilutions of bithionol in PBS, 0.2% DMSO (negative control), or 2% Triton-X 100 (positive control) in a 96-well plate. The 96-well plate was incubated at 37° C. for 1 h and then centrifuged at 500×g for 5 min. 50 μl of the supernatant was transferred to a fresh 96-well plate, and absorbance of supernatants was measured at 540 nm. Percent hemolysis was calculated using the following equation: (A₅₄₀ nm of compound treated sample−A₅₄₀ nm of 0.1% DMSO treated sample)/(A₅₄₀ nm of 1% Triton X-100 treated sample−A₅₄₀ nm of 0.1% DMSO treated sample)×100. All experiments were conducted in biological duplicate or triplicate and were independently repeated 3 times.

Transmission electron microscopy. S. aureus MW2 was grown to exponential phase (OD₆₀₀=˜0.4) at 37° C. Bacterial cultures were then treated with 10× MIC of bithionol or 0.1% DMSO (control) at 37° C. for 2 h. 1 mL of the treated bacterial samples were fixed with the same volume of a 2× fixative, a mixture of 5% glutaraldehyde, 2.5% paraformaldehyde and 0.06% picric acid in 0.2 M sodium cacodylate buffer (pH 7.4). Fixed cells were washed in 0.1 M cacodylate buffer and post-fixed with 1% Osmium tetroxide (OsO₄)/1.5% Potassium ferrocyanide (KFeCN₆) for 1 h. The fixed cells were washed twice in water, once in maleate buffer, and incubated in the maleate buffer containing 1% uranyl acetate for lh. Cells were then washed twice in water and subsequently dehydrated in an alcohol gradient series (10 min each; 50%, 70%, 90%, 2×10 min 100%). The cells were then incubated in propyleneoxide for 1 h, infiltrated overnight in a 1:1 mixture of propyleneoxide and Spurr's low viscosity resin (Electron Microscopy sciences, Hatfield, Pa.) and polymerized at 60° C. for 48 h. Ultrathin sections (about 60 nm) were cut on a Reichert Ultracut-S microtome (Leica Microsystem, Wetzlar, Germany), picked up onto copper grids, and stained with lead citrate. Micrographs of the cells were taken using a JEOL 1200EX transmission electron microscope (Harvard Medical School EM facility).

Giant unilamellar vesicles (GUVs) assay. GUVs were prepared by slightly modifying the electroformation method described previously. 1,2-dioleoyl-sn-glycero-3-phosphocholine (DOPC), 1,2-dioleoyl-sn-glycero-3-phospho-(1′-rac-glycerol) (DOPG), 1-palmitoyl-2-oleoyl-glycero-3-phosphocholine (POPC), cholesterol (ovine wool) and 1,2-dioleoyl-sn-glycero-3-phosphoethanolamine-N-(lissamine rhodamine B sulfonyl) (18:1 Liss Rhod PE) were purchased from Avanti Polar Lipids (Alabaster, Ala., USA). 4 mM lipid mixtures consisting of DOPC/DOPG/18:1 Liss Rhod PE (7:3:0.005) and POPC/cholesterol/18:1 Liss Rhod PE (7:3:0.005) were dissolved in chloroform respectively. Same procedure was used to prepare both kinds of GUVs. Indium tin oxide (ITO)-coated slides (50×75×1.1 mm, Delta Technologies, Loveland, Colo., USA) were coated with 20 ₁1.1 of the lipid mixture and dried in a vacuum chamber for 2 h to remove chloroform. An electroformation chamber was made by placing a 2 mm thick Teflon spacer between the two lipid-coated ITO slides. After adding 2 mL of 100 mM sucrose into the electrofomation chamber, the chamber was sealed with binder clips, and then connected to an AC field function generator. The swelling of the lipid bilayers was facilitated by applying an electric AC-field (10 Hz). The field strength was gradually increased from 0 to 4 kV/m for 15 min, and then was maintained constantly for 45 min. Detachment of GUVs from surfaces was conducted by reducing the AC-field from 10 Hz to 5 Hz for 15 min. The GUV suspension was slowly diluted (1:30) in a 110 mM glucose solution to induce sedimentation. The large vesicles were then separated from the small ones by centrifugation in 2 mL polypropylene microcentrifuge tubes at 3000 rpm for 2.5 min. The sediment was then collected and diluted (1:3) in a solution containing 1 volume of 100 mM sucrose and 6 volumes of 110 mM glucose. 49 μl of the diluted GUV suspension (˜100 vesicles) was added into a black, clear-bottom 384-well plate (Corning no. 3712). The plate was left in the dark at room temperature for 15 min until all GUVs settled on the bottom of the plates. After adding 1 μl of compound solution to a well (final compound concentration: 1× MIC), the GUVs were observed and imaged with an optical microscope equipped with fluorescence contrast and a digital camera (63x objectives, Axio Observer. A1 & AxioCam MRm, Zeiss, Germany). Images and movies are representative of three independent experiments.

All-atom molecular dynamics (MD) simulations. All-atom MD simulations based on the GROMACS package (8) were performed to investigate the interactions between bithionol or its analogs and a simulated bacterial or mammalian plasma membrane. Automated Topology Builder was employed to generate the topologies and parameters of bithionol and its 8 analogs that were compatible with GROMOS54a7 force field. Specifically, atomic charges of compounds were fitted to quantum mechanical electrostatic potentials at B3LYP/6-31G* level of theory; non-bonded parameters were refined against experimental solvation properties; bonded parameters were assigned using force constants estimated from Hessian (B3LYP/6-31G*). All of topologies and parameters can be downloaded freely in the internet with their molecule identities (molid) (Bithionol: 31957; Bitin-S: 33557; BT-OMe: 337704; BT-pBr: 337706; BT-pF: 337707; BT-oBr: 337708; BT-oF: 337709; BT-opBr: 337711; BT-opF: 337712). The plasma membrane of S. aureus was represented by a mixed lipid bilayer composed of 88 neutral-charged DOPC and 40 negatively-charged DOPG lipids (˜7:3 ratio) with dimensions of 5.96 nm×5.96 nm. This mixture of lipids is widely used to mimic anionic Gram-positive bacterial membranes and to investigate the mechanisms of action of membrane-active antimicrobials, such as daptomycin and antimicrobial peptides on S. aureus membranes. The mammalian plasma membrane was made up of POPC lipids with 30 molar-% cholesterol with dimensions of 5.51 nm×5.51 nm. The POPC lipid bilayers with different molar percentages of cholesterol (0%, 10%, 20%, and 30%) were also constructed to study the effects of cholesterol on the membrane selectivity of bithionol. The DOPC, DOPG, POPC lipids and cholesterol were modeled with Berger's lipid force field, which is an extensively validated all-atom lipid model for membrane-related simulations. Repetitions of simulations for different membrane compositions were performed to verify the robustness of results and mechanisms. Sodium ions were added into the simulation system to neutralize the negative charge of membranes. To visualize membrane attachment and penetration, bithionol and sodium ions are depicted as large spheres, phospholipids are represented as chains, and bonds in cholesterols are highlighted by thickened tubes. Water molecules are set to be transparent for clarity.

For enhanced computational efficiency, water molecules were represented by a polarization corrected simple point-charge SPC/E model. A geometric combining rule of Lennard-Jones potential was adopted for non-bonded interactions of bithionol and its analogs with lipids, ions and water. The fast smooth particle-mesh Ewald was used to calculate the long-rang electrostatic interactions. The system was modeled as an NPT ensemble, with periodic boundary conditions in all directions under constant pressure P (1 atm) and constant temperature T (300 K). The simulation box had an initial height of 12.3 nm, which was large enough to prevent the membrane and bithionol or its analogs molecules from interacting with their periodic images. The time step was fixed at 2 fs. After a 500 ns initial equilibration of solvated lipid systems, bithionol or its analogs was introduced into the water phase above the membrane. After 100 ns of re-equilibration, the bithionol and its analogs were released and their interactions with the membrane including attachment, penetration and equilibrium configurations were further simulated for 500 ns. The free energy profiles for the translocations of bithionol and its analogs were calculated by steered molecular dynamics, umbrella sampling, and the weighted histogram analysis methods, with the output giving the transfer energies and energy barriers that describe the feasibility (favorability and rate, respectively) of membrane penetration. The energy profile of penetration is a theoretical representation of an energetic pathway, as the bithionol and its analogs are translocated into a membrane, with two independent parameters: transfer energy and energy barrier. The transfer energy of penetration, which is defined as the energy conversion from outside solution to the equilibrium state inside membrane, describes the direction of translocation. The negative value of transfer energy that represents the energy decrease for penetration indicates that the embedment of bithionol or its analogs inside the membrane is energetically favorable. The energy barrier is calculated as the height of the peak along the pathway relative to the equilibrium state outside the membrane surface. The energy barrier is the least energy the bithionol or its analogs must possess to cross over the membrane surface, which governs the rate of penetration. A lower energy barrier corresponds to a faster and easier penetration. The thermal energy k_{B}T was used as the unit of energy in the simulations with T corresponding to room temperature (300 K). In equilibrium, the probability of a system being in a state with energy E is proportional to e^{-E/k}^{_{B}}^{T}. By using the k_{B}T as the measurement, the system stability could be explicitly compared at different equilibrium states. GridMAT-MD was used to calculate the thickness of bilayer membrane. With a grid of 20×20, thickness distributions of the bacterial membrane with or without embedment of bithionol were measured by a time average of 50 ns (500 frames). Configurational and thermodynamic analyses, including mass density profile of hydrophobic member interior and deuterium order parameters of lipid acyl chains, were performed to study the effects of the embedment of cholesterol in lipid bilayers. To valid the robustness of the proposed molecular mechanism subject to different force fields, we performed additional simulations of bithionol interacting with bacterial- or mammalian-mimetic membranes using the CHARMM force field. CHARMM-GUI was used to construct the 7DOPC/3DOPG and 7POPC/3 cholesterol lipid bilayers of dimensions 6.56 nm×6.56 nm×12.52 nm and 5.65 nm×5.65 nm×12.90 nm, respectively. The topology and parameters of bithionol were generated using the web server SwissParam by combining bonded parameters extracted from the Merck Molecular Force Field (MMFF) and nonbonded terms from the CHARMM. Other simulation protocols are the same as described above.

Deep-seated mouse thigh infection model. In vivo efficacy of bithionol alone or in combination with gentamicin against MRSA strain MW2 persisters was evaluated using a previously described neutropenic mouse thigh infection model with modifications. Six-week-old female CD-1 mice (20-25 g, Charles River Laboratories, Wilmington, Mass., USA) were rendered neutropenic by administering 150 mg/kg and 100 mg/kg of cyclophosphamide intraperitoneally (i.p.) at 4 days and 1 day before infection, respectively. On the day of infection, overnight culture of S. aureus MW2 were washed 3 times with sterile saline and diluted to ˜10⁴ CFU/mL in saline. 50 μl of the diluted culture was injected to the right thigh of each mouse. Bithionol was dissolved in Kolliphor EL (Sigma-Aldrich, St Louis, Mo., USA)/ethanol 1:1 and then diluted 1:10 in saline to a final concentration of 30 mg/kg. At 24 h post-infection, groups of mice (n=10 biological independent animals) were treated with 30 mg/kg gentamicin subcutaneously (s.c.), 25 mg/kg vancomycin i.p., 30 mg/kg bithionol i.p., 30 mg/kg gentamicin s.c. in combination with 30 mg/kg vancomycin i.p., or 30 mg/kg gentamicin s.c. in combination with 30 mg/kg bithionol i.p. every 12 h for 3 days. Control mice were injected with 100 μl of 10% Kolliphor EL/ethanol in saline i.p. every 12 h for 3 days. Mice were euthanized at 96 h post-infection. Blood was collected by cardiac puncture for evaluating hepatic and renal toxicities, and the infected thighs were aseptically excised, weighed, and stored at 4° C. until homogenization. The levels of alanine aminotransferase and blood urea nitrogen were analyzed with commercially available kits, following the manufacturer's protocol (Pointe Scientific, Canton, Mich., USA) to evaluate hepatic and renal toxicities. Thighs were homogenized in PBS, serially diluted with PBS, and spot-plated on TSA plates. After incubating the TSA plates at 37° C. overnight, the number of colonies was enumerated to calculate CFU/g thigh tissue. This study and all experiments involving mice were performed in accordance with guidelines approved by the Rhode Island Hospital Institutional Animal Care and Use Committee (RIFT IACUC). Statistical significance among each group in animal studies was analyzed by one-way ANOVA with post-hoc Tukey test using GraphPad Prism 7 (GraphPad Software, La Jolla, Calif., USA).

### Example 1

### Chemical Synthesis of Exemplified Compounds

Synthetic schemes describing chemical synthesis of the exemplified compounds are shown in FIGS. 5, 17, and 18. Chemical structures of the exemplified compounds are also depicted in FIGS. 5, 17, and 18.

General synthetic procedures for bithionol analogs. To a flame-dried round-bottom flask charged with a Teflon-coated stir bar and AlCl₃ (1.0 equiv.), CH₂Cl₂ was added (0.5 M) under inert atmosphere and left to stir. Substituted phenol (1.0 equiv.) was added portion-wise at 0° C., followed by slow addition of thionyl chloride (1.0 equiv.) over 30 minutes. The mixture was warmed to ambient temperature and monitored by TLC (35% EtOAc in hexanes). Upon consumption of starting materials, the reaction mixture was poured into ice water and separated with EtOAc. The combined organic layers were washed with brine, dried (MgSO₄), filtered, and concentrated in vacuo to yield crude sulfoxide product. Crystallization was achieved from boiling toluene and was checked by direct-injection for LRMS in negative ion mode. Sulfoxide material was carried through to the next step without additional characterization.

To a round-bottom flask charged with a Teflon-coated stir bar and sulfoxide, material from the previous step (1.0 equiv.), was diluted in glacial acetic acid (0.1 M). Zinc dust (10.0 equiv.) was added to the mixture under inert atmosphere and left to stir. The reaction was refluxed at 100° C. for 4 hours and monitored by TLC (35%

EtOAc in hexanes). Upon completion, the reaction mixture was cooled to ambient temperature and passed through a plug of celite. The resulting solution was diluted in EtOAc and washed with a saturated solution of sodium bicarbonate (2×). The combined organic layers were further washed with water (3×) and brine (3×), dried (MgSO₄), filtered, and concentrated in vacuo to yield crude sulfide product. Crude product was diluted in a solution of methanol and CH₂Cl₂ (1:1) and checked by direct-injection for LRMS in negative ion mode. Crystallization was achieved from boiling CH₂Cl₂ and subjected for characterization.

6,6′-thio-bis-(2,4-dichlorophenol) [bithionol]: Crystallization was achieved from hot CH₂Cl₂to yield 3.75 g (10.5 mmol) of a white crystalline powder in 73% yield over two steps. m.p.=184-186° C. HRMS (ESI-TOF) m/z: calculated for C₁₂H₅Cl₄O₂S [(M−H)⁻]352.87698, observed 352.87690 (Δ=0.08 mmu). ¹HNMR (400 MHz, (CD₃)₂CO): δ 9.08 (s, 2H), 7.45 (dd, J=4.9, 2.5 Hz, 2H), 7.13 (dd, J=4.8, 2.5 Hz, 2H) ppm. ¹³CNMR (125 MHz, (CD₃)₂CO): δ 151.91, 131.24, 130.09, 125.53, 124.05, 122.78 ppm. IR (thin film) vₘₐₓ: 3391, 3338 cm⁻¹.

6,6′-thio-bis-(4-bromo-2-chlorophenol) [BT-pBr]: Isolated 249 mg (0.560 mmol) of a pale lavender powder in 54% yield over two steps (recrystallization solvent, CH₂Cl₂). m.p.=200-202° C. HRMS (ESI-TOF) m/z: calculated for C₁₂H₅B r₂Cl₂O₂S [(M−H)⁻]440.77595, observed 440.77643 (Δ=0.48 mmu). ¹HNIVIR (400 MHz, (CD₃)₂CO): δ 7.46 (s, 2H), 7.36 (s, 2H) ppm. IR (thin film) vₘₐₓ: 3063 cm⁻¹.

6,6′-thio-bis-(2-chloro-4-fluorophenol) [BT-0]: Isolated 225 mg (0.696 mmol) of an off-white powder in 51% yield over two steps (recrystallization solvent, CH₂Cl₂). m.p.=140-142° C. HRMS (ESI-TOF) m/z: calculated for Cl₂H₅Cl₂F₂O₂S [(M−H)⁻]320.93609, observed 320.93608 (Δ=0.01 mmu). ¹HNMR (400 MHz, (CD₃)₂CO): δ 8.64 (br s, 2H), 7.25 (dd, J=8.1, 3.0 Hz, 2H), 6.90 (dd, J=8.3, 3.0 Hz, 2H) ppm. IR (thin film) vₘₐₓ: 3340 cm⁻¹.

6,6′-thio-bis-(2-bromo-4-chlorophenol) [BT-oBr]: Isolated 325 mg (0.730 mmol) off-white powder in 69% yield over two steps (recrystallization solvent, CH₂Cl₂). m.p.=178-180° C. HRMS (ESI-TOF) m/z: calculated for C₁₂H₅Br₂Cl₂O₂S [(M−H)⁻]440.77595, observed 440.77607 (Δ=0.12 mmu). ¹HNMR (400 MHz, (CD₃)₂CO): δ 8.92 (br s, 2H), 7.59 (d, J=2.1 Hz, 2H), 7.18 (d, J=1.3 Hz, 2H) ppm. IR (thin film) vₘₐₓ: 3355, 3064 cm⁻¹.

6,6′-thio-bis-(4-chloro-2-fluorophenol) [BT-oF]: Isolated 170 mg (0.525 mmol) of a white powder in 65% yield over two steps (recrystallization solvent, CH₂Cl₂). m.p.=159-161° C. HRMS (ESI-TOF) m/z: calculated for Cl₂H₅Cl₂F₂O₂S [(M−H)⁻]320.93609, observed 320.93612 (Δ=0.03 mmu). ¹HNMR (400 MHz, (CD₃)₂CO): δ 8.61 (br s, 2H), 7.27 (dd, J=8.1, 3.0 Hz, 2H), 6.92 (dd, J=8.3, 3.0 Hz, 2H) ppm. IR (thin film) vₘₐₓ: 3301 cm⁻¹, 3081 cm⁻¹.

6, 6′-thio-bis-(2, 4-dibromophenol) [BT-opBr]: Isolated 67.6 mg (0.151 mmol) of an off-white powder in 58% yield over two steps (recrystallization solvent, CH₂Cl₂). m.p.=164-166° C. HRMS (ESI-TOF) m/z: calculated for C₁₂H₅Br₄O₂S [(M−H)⁻]528.67492, observed 528.67482 (Δ=0.10 mmu). ¹HNMR (400 MHz, (CD₃)₂CO) (rotamers): δ 9.36 (br, s, 2H), 7.29-7.25 (m, 2H), 6.99-7.00 (m, 2H) ppm. IR (thin film) vₘₐₓ: 3364, 3057, 2921 cm⁻¹.

6,6′-thio-bis-(2,4-difluorophenol) [BT-opt]: Isolated 163 mg (0.558 mmol) of a beige powder in 52% yield over two steps (recrystallization solvent, CH₂Cl₂). m.p.=127-130° C. HRMS (ESI-TOF) m/z: calculated for C₁₂H₅F₄O₂S [(M−H)⁻]288.99519, observed 288.99519 (Δ=0.00 mmu). ¹HNMR (400 MHz, (CD₃)₂CO): δ 8.99 (s, 2H), 7.06 (ddd, J=10.6, 8.5, 3.0 Hz, 2H), 6.75 (ddd, J=8.6, 3.0, 1.9 Hz, 2H) ppm. IR (thin film) vₘₐₓ: 3317, 3091 cm⁻¹.

bis-(3,5-Dichloro-2-methoxyphenyl)-sulfane [BT-OMe] (See FIG. 18): To a solution of bithionol (100 mg, 0.280 mmol, 1.0 equiv.) in DMF (0.3 M) was added K₂CO₃ (165 mg, 1.19 mmol, 4.25 equiv.) and Me₂SO₄ (94 μL, 0.980 mmol, 3.5 equiv.). The resulting mixture was stirred overnight at room temperature and monitored by TLC (35% EtOAc in hexanes). Upon completion, the reaction was quenched water and separated from EtOAc (3 ×). The combined organic layers were washed with water (2×) and brine (3 ×), dried (MgSO₄), filtered, and concentrated in vacuo to afford clear, pale yellow oil. Purification by column chromatography (silica gel, 20% EtOAc in hexanes) yielded 87 mg (0.227 mmol) of BT-OMe as clear, colorless oil in 81% yield. HRMS (ESI+TOF) m/z: calculated for C₁₄H₁₁O₂Cl₄S [(M+H)⁺]382.92284, observed 382.92295 (Δ=0.11 mmu). ¹HNMR (400 MHz, (CD₃)-₂CO): δ 7.51 (d, J=2.2 Hz, 2H), 17.15 (d, J=2.2 Hz, 2H), 3.90 (s, 6H) ppm. IR (thin film) vₘₐₓ: 3070, 2937 cm⁻¹.

### Example 2

### Bithionol Shows Bactericidal Activity against both Antibiotic-Resistant S. aureus and S. aureus Persister Cells

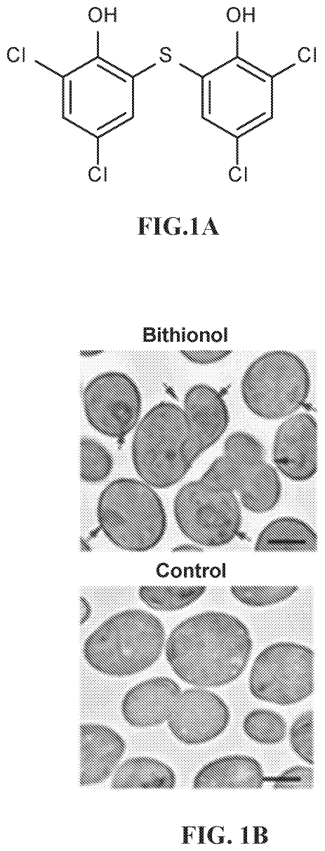

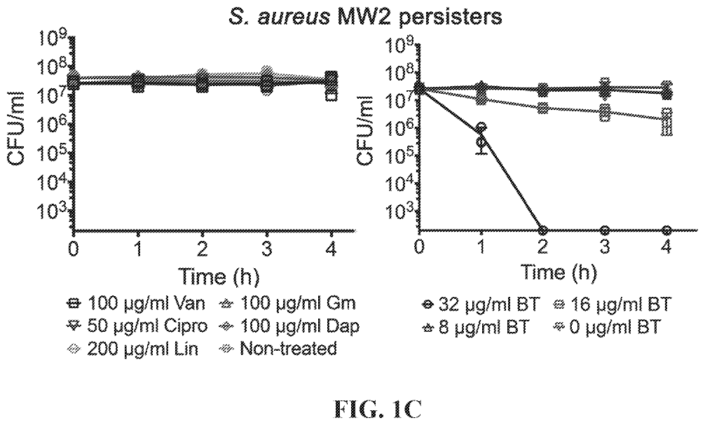

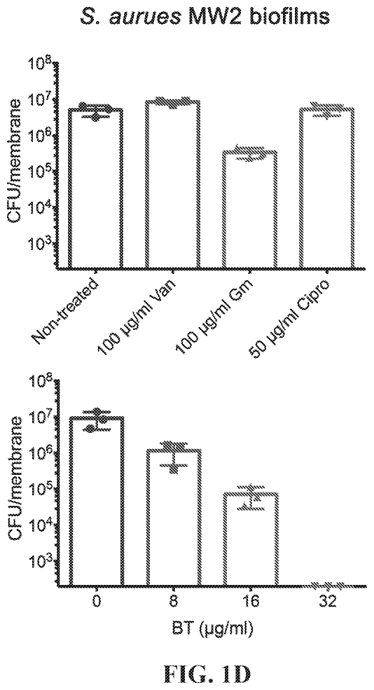

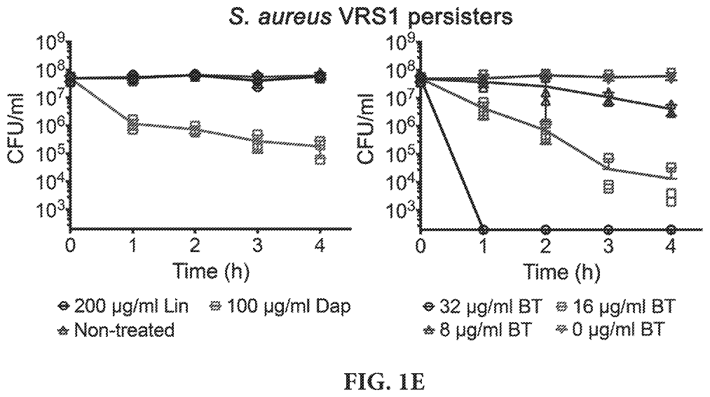

Bithionol (FIG. 1A) was previously described as an antimicrobial agent with a minimal inhibitory concentration (MIC) of approximately 8-15 μg/mL against Gram-positive bacteria including S. aureus, Bacilius subtilis, and Enterococcus faecium, and an MIC of >100 μg/mL against Gram-negative bacteria including Escherichia coli and Shigella dysenteriae. Bithionol also exhibited antimicrobial activity against a panel of 27 Gram-positive pathogens, including vancomycin-resistant S. aureus (VRSA) and vancomycin- and daptomycin-resistant Enterococcal strains, although in our hands bithionol was more potent than previously reported with minimum inhibitory concentrations of 0.5-2 μg/mL (FIG. 6). Daptomycin exhibited comparable MICs ranging from 0.5-8 μg/mL against the 18 S. aureus strains included in the panel of tested strains (FIG. 6). Compared to the Gram-positive strains, bithionol exhibited relatively weak antimicrobial activity against Gram-negative pathogens (MICs 16−>64 μg/mL) (FIG. 6).

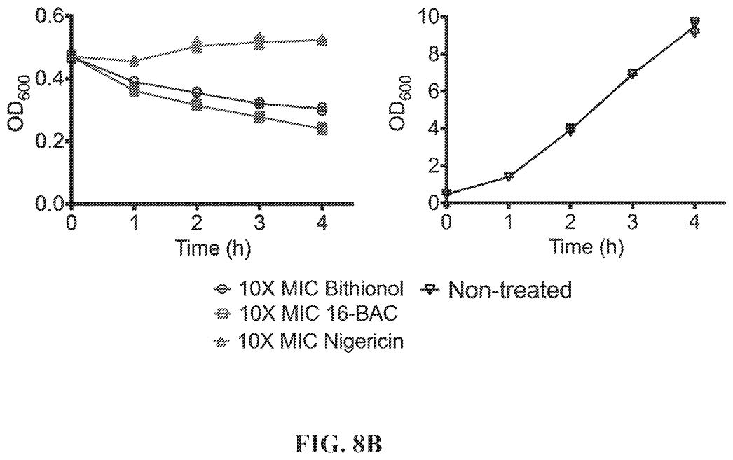

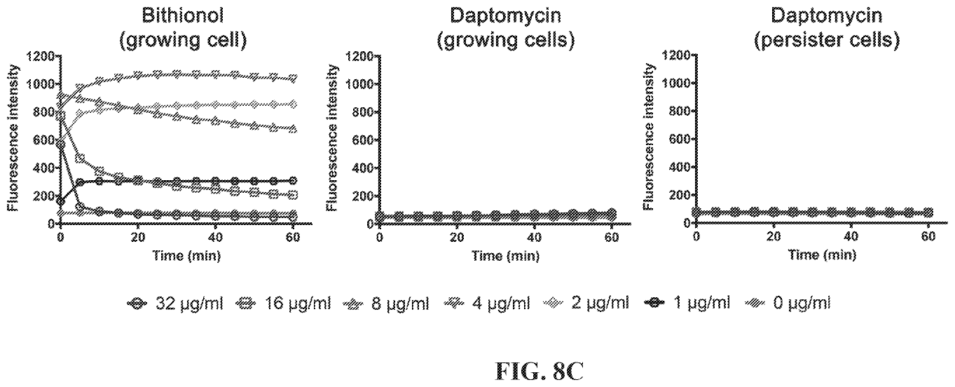

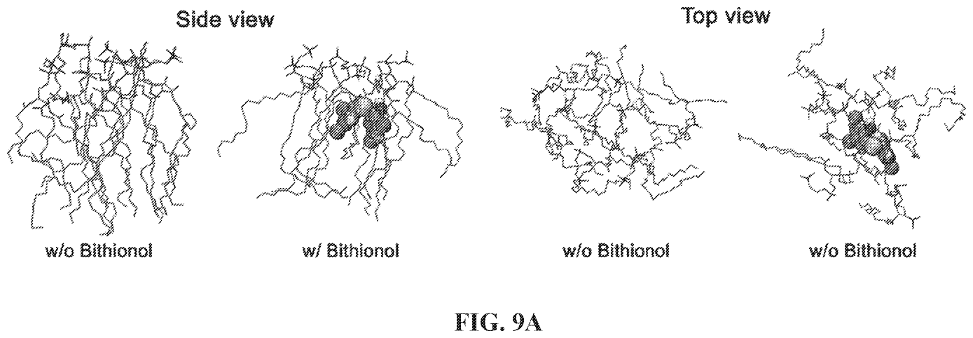

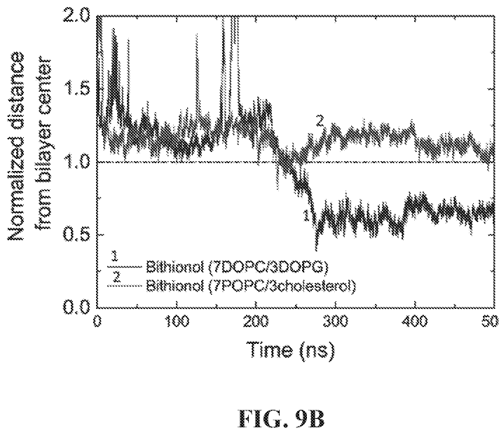

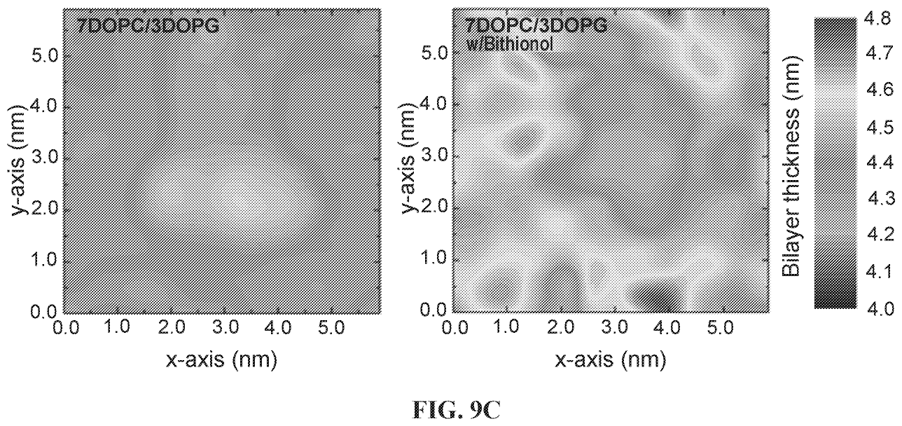

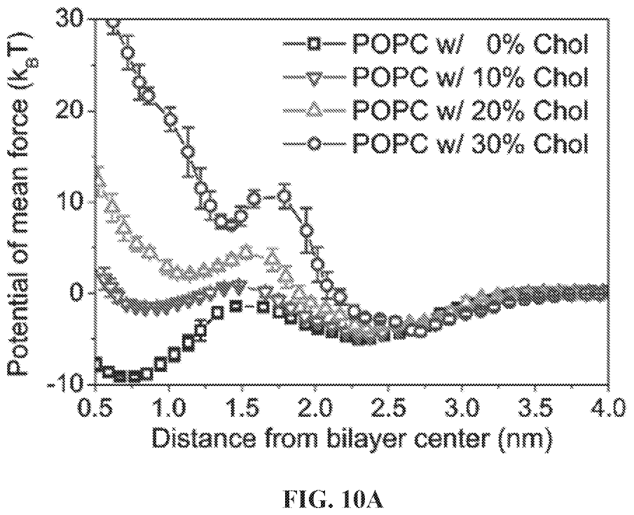

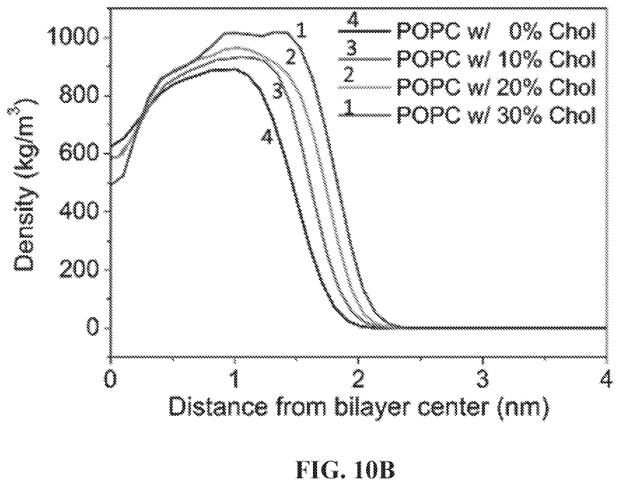

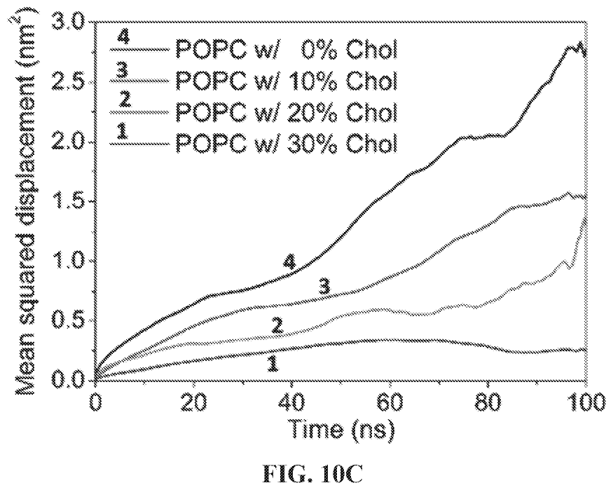

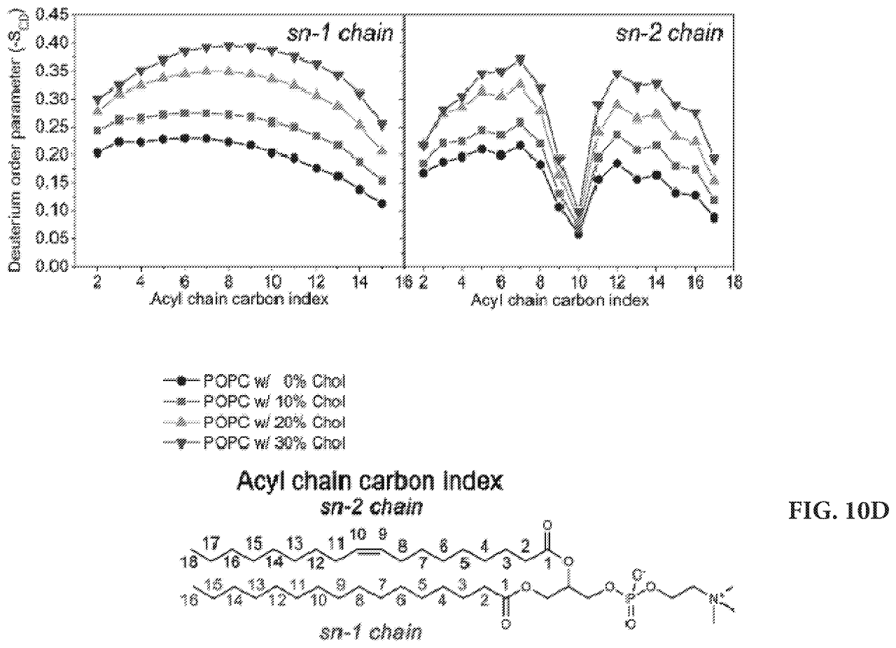

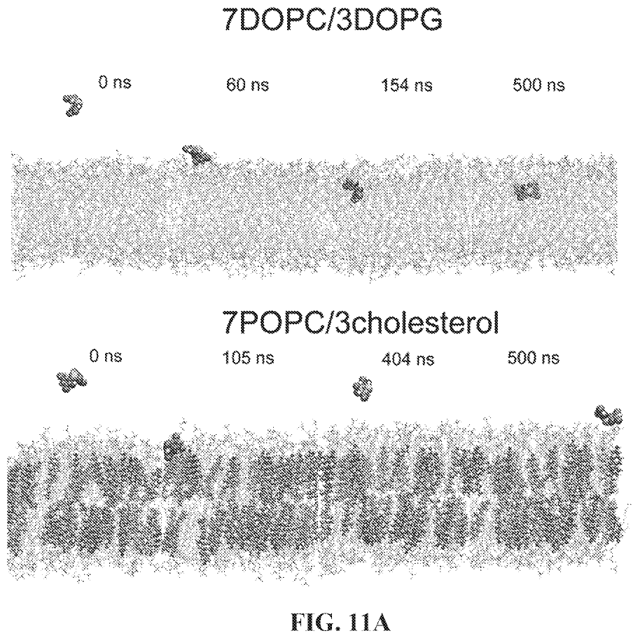

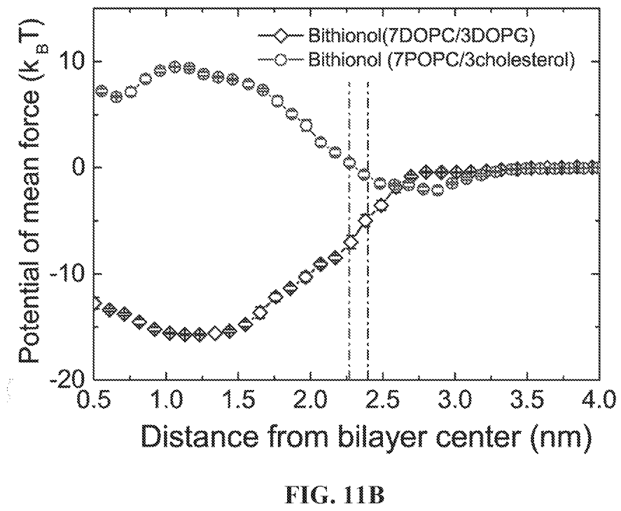

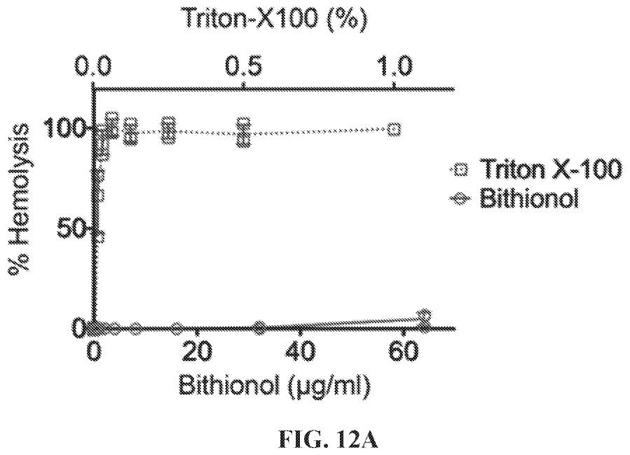

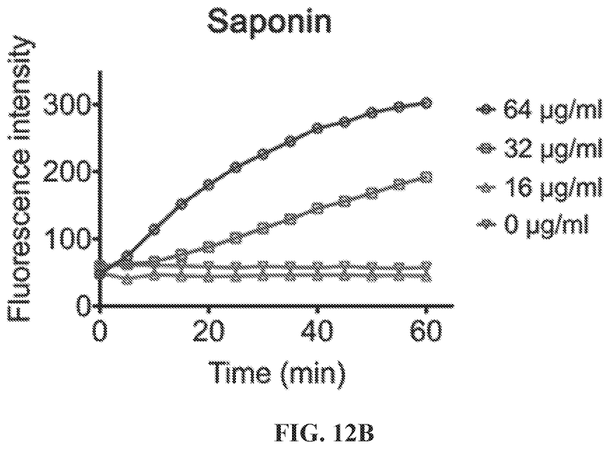

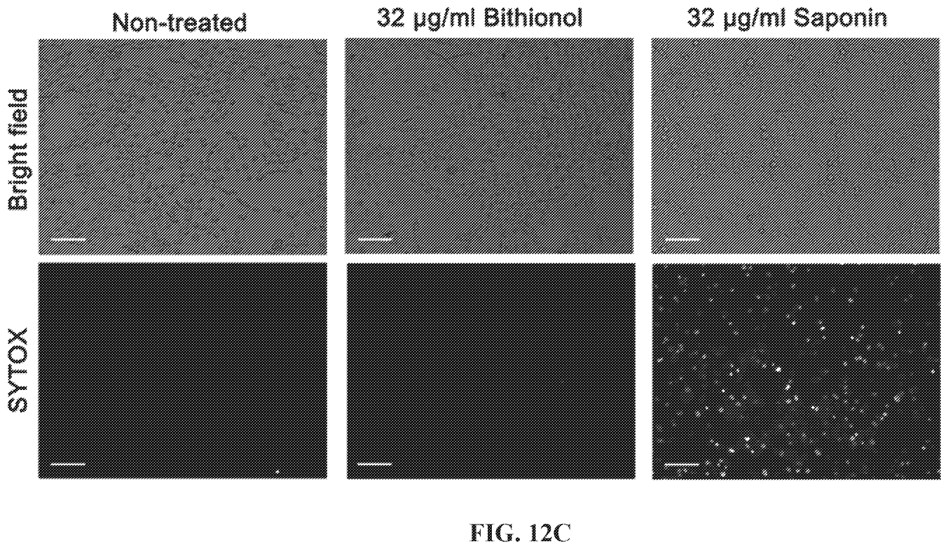

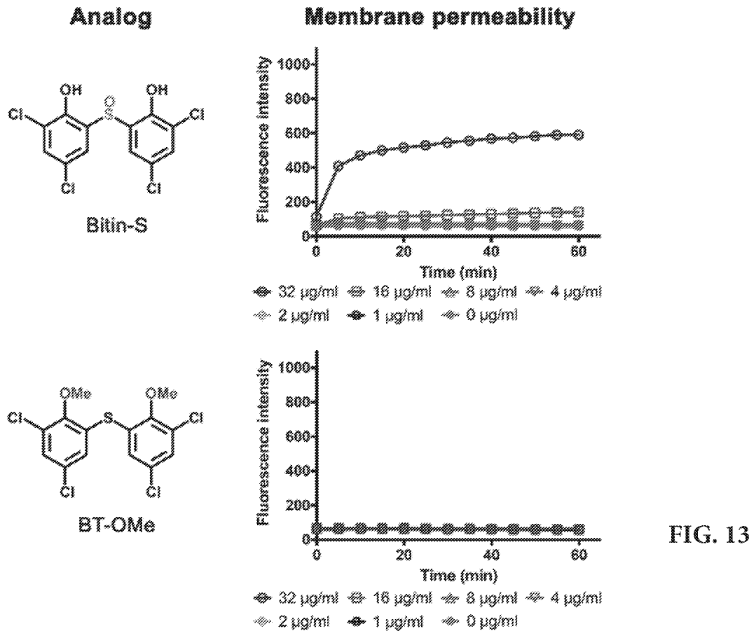

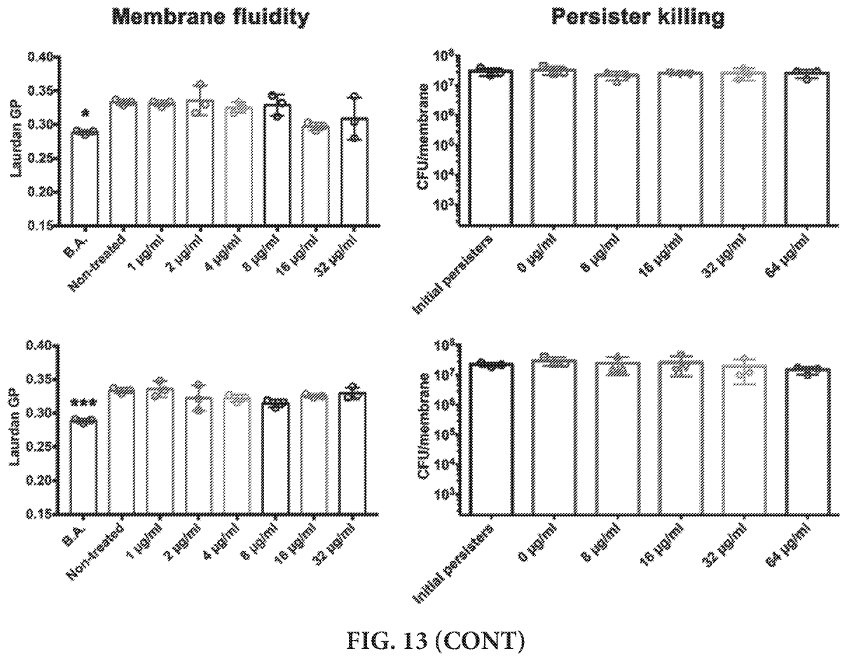

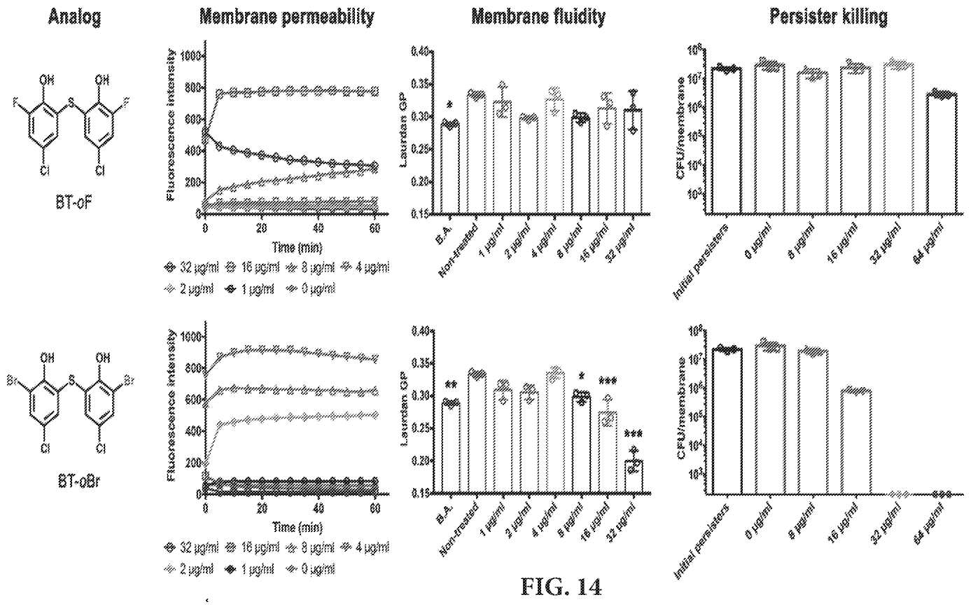

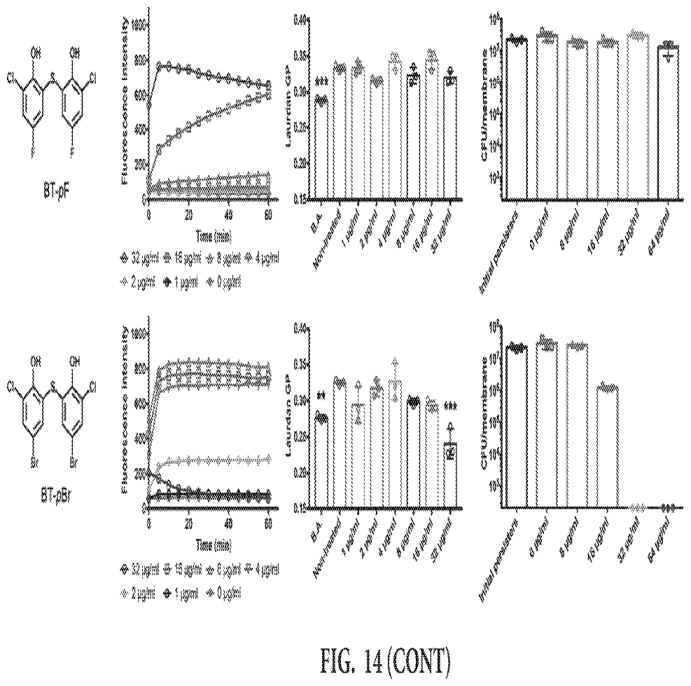

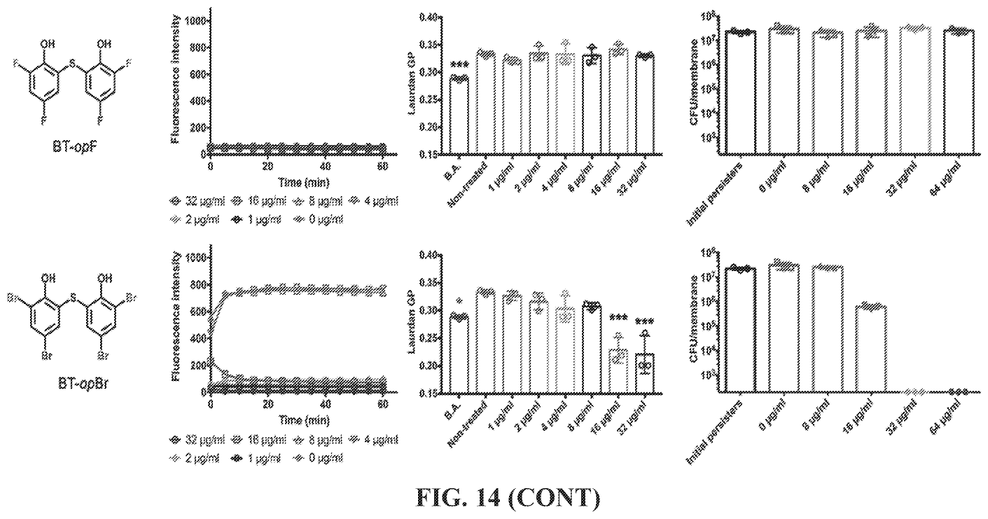

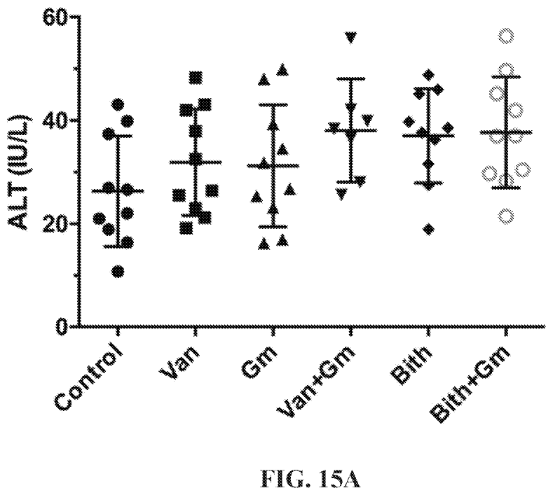

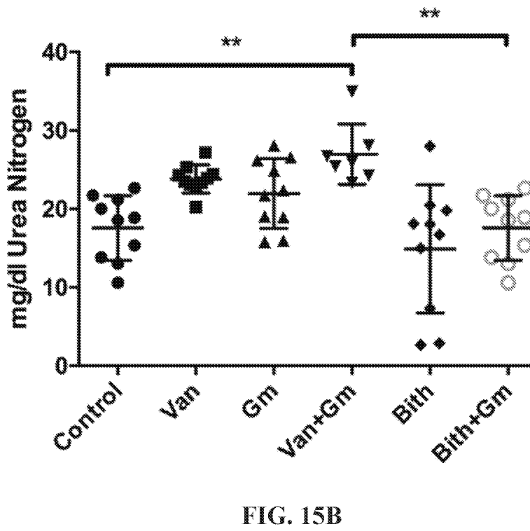

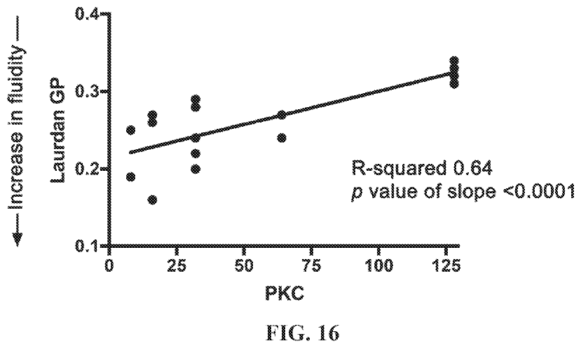

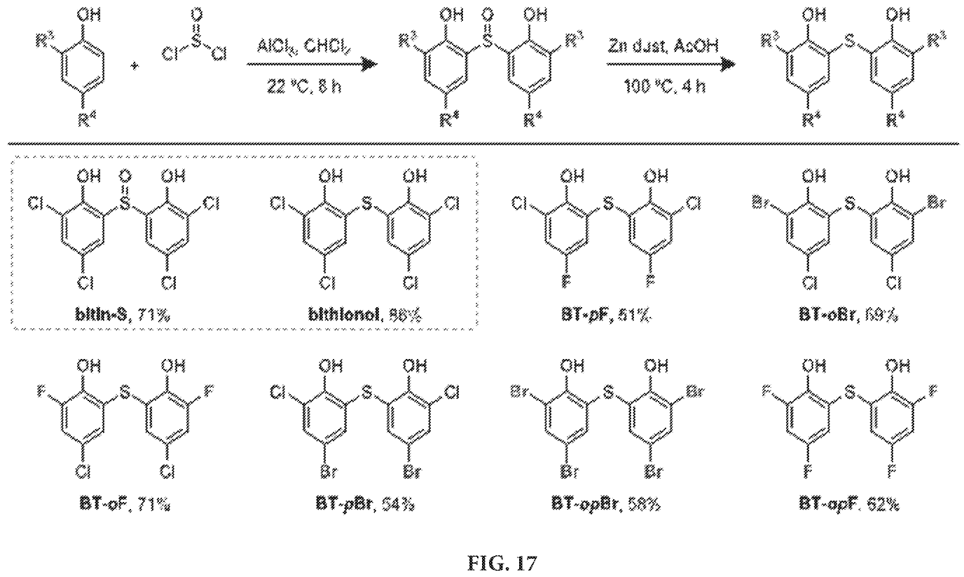

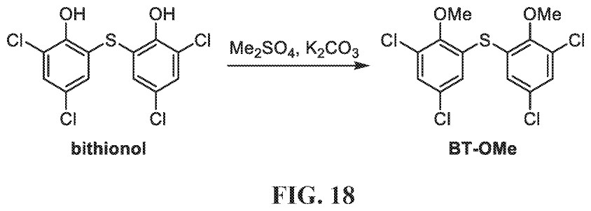

In contrast to a previous study that described bithionol as bacteriostatic, the present results show that bithionol eradicated ˜10⁷ CFU/mL exponential-phase S. aureus MW2 within 3 h at 10 μg/mL (10× MIC) (FIG. 8). The rate of killing was comparable to daptomycin (at 10× MIC) but significantly faster than the killing kinetics of vancomycin (at 10× MIC), two antibiotics of “last resort” for S. aureus infections (FIG. 8). Further, unlike bacteriostatic antimicrobials like nigericin, 10 μg/mL bithionol induced a time-dependent decrease in optical density of S. aureus cells comparable to the antiseptic detergent benzyldimethylhexadecylammonium chloride (16-BAC, FIG. 8), indicating that bithionol has bacteriolytic activity.

Indeed, transmission electron micrographs (TEM) showed that 10× MIC bithionol disrupts MRSA membranes, causing the intracellular formation of mesosome-like structures, abnormal cell divisions, and cell lysis (FIG. 8), which were previously observed in the S. aureus cells treated with membrane-active antimicrobials.

Although MRSA persisters displayed a high level of tolerance to 100× MIC of several conventional antibiotics including daptomycin and linezolid, bithionol killed stationary-phase MRSA persisters and biofilm persisters in a dose- and time-dependent manner, and completely eradicated them at 32 μg/mL (32× MIC) within 2 h and 24 h, respectively (FIGS. 1C, D). Furthermore, bithionol was also highly efficacious at eradicating vancomycin-resistant S. aureus (VRSA) strain VRS1 persisters, whereas linezolid and daptomycin exhibited no and nominal activity, respectively (FIG. 1E). Collectively, these results demonstrate that bithionol is a bactericidal agent effective against both multidrug-resistant S. aureus strains and their persister cells.

### Example 3

### Bithionol Interacts with and Disrupts the Bacterial Mimetic Pipid Bilayer

Because bithionol was indentified as a compound that permeabilized MRSA cells, all-atom molecular dynamics (MD) simulations were performed of bithionol interacting with simulated bacterial membranes to elucidate a potential mechanism of action. The topology and parameters of a bithionol molecule for GROMOS54a7 force field was generated by Automated Topology Builder, which has been widely used to model bioactive small molecules. Previously established model of 1,2-dioleoyl-sn-glycero-3-phosphocholine (DOPC)/1,2-dioleoyl-sn-glycero-3-phospho-(1′-rac-glycerol) (DOPG) was used at a 7:3 ratio to simulate negatively-charged S. aureus membranes (see Materials and Methods). Bithionol is initially recruited to the membrane surface by the binding of polar hydroxyl and chlorine groups to hydrophilic lipid heads (FIG. 2A). After several hundred nanoseconds of sustained attachment, bithionol penetrates into the membrane interior, maximizing interactions between nonpolar benzene rings and hydrophobic lipid tails. After penetration, bithionol embeds vertically in the outer leaflet of the lipid bilayer (FIG. 2A). Moreover, the insertion of even a single bithionol molecule causes a substantial local increase in lipid bilayer disorder (FIGS. 9A, C). These MD simulations demonstrate that the polarity of branch groups and the hydrophobicity of core rings play important roles in membrane attachment and penetration.

The effects of bithionol on lipid bilayers were also studied using biomembrane-mimicking giant unilamellar vesicles (GUVs) consisting of a single lipid bilayer with a diameter of 10-100 μm. GUVs were constructed using DOPC/DOPG lipids at a 7:3 ratio as in the MD simulations. Lipid aggregates formed on the surfaces of the GUVs exposed to 1 μg/mL bithionol and at 10 μg/mL the GUVs burst (FIG. 2C), indicating that bithionol interacts with and disrupts a bacterial mimetic lipid bilayer.

### Example 4

### Bithionol Induces Rapid Membrane Permeabilization and an Increase in Membrane Fluidity

SYTOX Green permeability assay was used to evaluate the membrane disrupting activity of bithionol. Unlike daptomycin, bithionol induced dose-dependent membrane permeability in both exponential-phase MRSA MW2 cells and stationary-phase MRSA MW2 persisters (FIG. 2D, FIG. 8C). The fluorescence intensity was maximized at 4 μg/mL and then decreased at concentrations above 8 μg/mL bithionol, likely due to bacterial lysis at the higher concentrations as shown by TEM (FIG. 1B). The dose-dependent killing kinetics of bithionol correlated with its dose-response effects on membrane permeability (FIGS. 1C, 2D and FIG. 8C), suggesting that the bactericidal activity of bithionol results from its membrane-activity.

We further investigated how insertion of bithionol into lipid bilayers causes membrane disruption. In physiological conditions, bacterial membrane lipid bilayers are mostly in a liquid-crystalline phase, which allows the membranes to properly function as a protective barrier and to stabilize membrane-bound proteins. Since homeostasis of membrane fluidity is crucial for survival, bacteria regulate membrane fluidity (viscosity) by adjusting their phospholipid compositions in response to stresses, such as temperature, osmolarity and pH. Insertion of compounds into membrane lipid bilayers are known to cause dramatic changes in membrane fluidity that can result in passive permeabilization, loss of membrane protein functions, leakage of cellular components, and bacterial death. It was tested whether the insertion of bithionol alters MRSA membrane fluidity by utilizing the membrane fluidity-sensitive dye Laurdan. Laurdan exhibits a fluorescence emission wavelength shift depending on the amount of adjacent water molecules. Since the extent of water molecule penetration into lipid bilayers is determined by lipid packing density and lipid bilayer fluidity, bacterial membrane fluidity can be estimated by the quantification of the fluorescence shift using Laurdan generalized polarization (GP)=(I440-I490)/I440+I490) (FIG. 2E) ranging from −1 (most fluid and disordered) to +1 (most rigid and ordered). As shown in FIG. 2E, bithionol at concentrations over 8 μg/mL induced a significant decrease in Laurdan GP in S. aureus MW2, similar to 50 mM benzyl alcohol (BA), a known membrane fluidizer, indicating that the penetration of bithionol into lipid bilayers increases MRSA membrane fluidity. Consistent with these results, bithionol concentrations greater of 16 μg/mL exhibited significant killing activity against MRSA MW2 persisters and 32 μg/mL completely eradicated them (FIG. 1C).

### Example 5

### Bithionol does not Penetrate Mammalian Membranes

Generally, membrane-active agents tend to damage both bacterial and mammalian lipid bilayers; however, bithionol has been clinically approved for treating helminthic infections and mammalian cell lytic activity has not been reported. Thus, we investigated whether bithionol's membrane disrupting activity is significantly more pronounced towards bacterial membranes compared to mammalian cell membranes. The lipid components of mammalian membranes mainly include phospholipids, sphingolipids, and cholesterol, the ratios of which vary depending on the types of cells, tissues, and/or organelles. Despite the complexity of their lipid composition, mammalian membrane lipid bilayers can be modeled as a simplified bilayer composed of the zwitterionic lipid phosphocholine (PC) mixed with cholesterol ranging from 20 to 50 mol %. Indeed, the simplified lipid bilayer model consisting of 1,2-palmitoyl-oleoyl-sn-glycero-3-phosphocholine (POPC)/cholesterol has being widely used to investigate the interactions between membrane-active antimicrobials and mammalian membrane lipid bilayers.

First, MD simulations were carried out of bithionol interacting with model mammalian cell membranes consisting of mixtures of POPC and cholesterol. At a POPC/cholesterol ratio of 7:3, MD simulations showed that bithionol fails to penetrate the POPC/cholesterol bilayer (FIGS. 2A, B and FIG. 9B). Moreover, the energy barrier and transfer energy for bithionol increases with increasing percentages of cholesterol (FIG. 10A). The failure of bithionol to penetrate the POPC/cholesterol membranes is consistent with configurational and thermodynamic analyses, demonstrating that cholesterol condenses the hydrophobic region of the membrane (FIG. 10B) and decreases membrane fluidity (FIG. 10C). The failure of bithionol to penetrate the POPC/cholesterol bilayer is also consistent with the observation that the presence of cholesterol results in a more ordered alignment of the membrane lipids (FIG. 10D). The ability for bithionol to discriminately penetrate the bacterial-mimetic over mammalian-mimetic lipid bilayer (FIGS. 2A, B) was consistently observed in another MD simulations using the CHARMM force field (FIG. 11). These results suggest that bithionol should exhibit a relatively high degree of selectivity towards bacterial membranes, mainly due to the presence of cholesterol in mammalian cell membranes but its absence in bacterial membranes.

Consistent with the MD simulations, bithionol did not cause observable effects on GUVs formed with 7POPC/3 cholesterol (FIG. 2C). Moreover, as predicted by the results obtained with the MD simulations and GUVs, bithionol exhibited relatively little hemolytic activity against human erythrocytes with an HC₅₀>64 μg/mL (FIG. 12A) and did not induce SYTOX Green membrane permeabilization of HKC-8 human renal proximal cells up to 64 μg/mL (FIG. 2D and FIG. 12B, C).

### Example 6

### Testing of New Compounds Synthesized in Example 1

MD simulations predicted that two key elements for membrane activity of bithionol are the initial binding to the membrane surface using phenolic hydroxyl groups and membrane perturbation induced by chlorinated benzene (FIG. 2A). 7 new compounds were synthesized (FIGS. 5, 13, 14, 17). Bitin-S is a previously described sulfoxide derivative of bithionol. The effect of binding affinity on antimicrobial and membrane activity was tested using bitin-S and the bithionol methoxy analog, BT-OMe (FIGS. 5, 18). The polar sulfinyl group of bitin-S provides additional hydrophilic interactions with lipid heads. The oxidized derivative bitin-S exhibited decreased antimicrobial activity (MIC 8 μg/mL) and reduced membrane activity (FIGS. 5 and 13). Reduced polarity by substituting methoxy groups for the two hydroxyl groups (BT-OMe, FIGS. 5, 13, 18) resulted in complete nullification of both antimicrobial and membrane activity (FIGS. 5, 13), indicating that the phenolic hydroxyl groups are critical for antimicrobial activity.

To test whether the size and polarization of the inserted molecules can affect the extent of membrane perturbation, alternative halogens were used for chlorine. Indeed, the replacement of chlorine with fluorine results in slightly reduced membrane and antimicrobial activities as seen with compounds BT-oF, BT-pF, and BT-opF, while substitution with bromine shows similar membrane and antimicrobial activities to bithionol demonstrated by BT-oBr, BT-pBr, and BT-opBr (FIGS. 5 and 14). It is likely that the polarity of the C-F bond in the fluoro derivatives may increase the hydrophilicity of the aryl rings, thus slightly decreasing membrane permeability. The bromine derivatives showed increased energy barrier values of 2-3 kBT (FIG. 7), indicating that the larger bromine atoms may cause more membrane perturbation than the smaller chlorine and fluorine atoms, although the bromines may be slightly disadvantageous for initial binding and penetration.

### Example 7

### Bithionol in Combination with Gentamicin Shows Efficacy in a Mouse Deep-Seated MRSA Infection Model

The concentration of bithionol required to eradicate 5×10⁷ CFU/mL MRSA persisters is 32 μg/mL, which is higher than desired. The persister-killing efficiency of bithionol could be increased through co-treatment with another antibiotic. Relatively low concentrations of bithionol (8 μg/mL) do not lead to a significant decrease in the viability of MRSA persisters (FIGS. 1C, D), 8 μg/mL bithionol combined with as little as 2 μg/mL gentamicin completely eradicated MRSA MW2 stationary-phase persisters cells (FIG. 3A). Similarly, 8 μg/mL bithionol +16 μg/mL gentamicin eradicated biofilm persisters (FIG. 3B).

The efficacy of bithionol in combination with gentamicin was further tested in an MRSA mouse thigh infection model, which mimics human deep-seated chronic infections. Consistent with a previous study, vancomycin, gentamicin, or a combination of the two did not significantly reduce MRSA CFUs in the mouse thigh (FIG. 3C), suggesting that the infecting bacterial cells are persisters. Although 30 mg/kg bithionol alone showed no effect on the viability of MRSA persisters, 30 mg/kg bithionol combined with 30 mg/kg gentamicin killed ˜90% of the MRSA persister cells (p<0.001) (FIG. 3C). The hepatic and renal toxicity of bithionol were evaluated in the mice in the experiments described in FIG. 3C by measuring serum levels of alanine aminotransferase (ALT) and blood urea nitrogen (BUN) (FIG. 15). Although the combination of vancomycin and gentamicin significantly increase BUN levels (p<0.01), the combination of bithionol and gentamicin increased neither.

### Example 8

### The Killing of MRSA Persisters is Achieved by Sufficiently Severe Membrane Disruption Featured as Increased Membrane Fluidity

The ability of a compound to permeabilize MRSA persister cell membranes is a good proxy for the ability of a compound to kill the persisters. However, some compounds that permeabilize MRSA persister cell membranes to SYTOX Green, such as bitin-S, BT-oF, or BT-pF, do not kill them (FIGS. 5, 13, 14). This was also observed for another membrane-active antimicrobial, nTZDpa, which has a completely different chemical structure from bithionol. For instance, nTZDpa-analogs 6 and 11 (FIG. 4B) induced rapid SYTOX Green permeabilization, but did not affect the viability of MRSA persisters. These results indicate that SYTOX Green membrane permeability alone is not a reliable indicator of anti-persister activity. Interestingly, only compounds leading to a significant increase in membrane fluidity killed MRSA persisters (FIG. 5, FIG. 4A and FIGS. 13, 14). Consistent with these data, a recent study reported that the antibiotic rhodomyrtone causes increased membrane fluidity in Bacillus subtilis and kills its persister cells.

Based on these results with bithionol and the newly synthesized exemplified compounds, there is likely a general correlation between alternation in membrane fluidity and anti-persister potency. To test this hypothesis, the effect of nTZDpa and 11 nTZDpa derivatives were measured on MRSA membrane fluidity (FIG. 4B). nTZDpa and its 11 analogs (FIG. 4B) are membrane-active antimicrobials that cause SYTOX Green membrane permeabilization of MRSA persisters. Consistent with the bithionol analogs, only nTZDpa analogs having anti-persister potency showed increased membrane fluidity at 32 μg/mL. Specifically, nTZDpa analogs 6 and 11, which had no anti-persister potency, did not result in alternation in membrane fluidity (FIG. 4B). Furthermore, substantial correlation was observed between the concentration of compound required for persister killing and the amount of induced membrane fluidity as measured by Laurdan GP for all 21 tested compounds at 32 μg/mL (R² 0.64, p of slope<0.0001, FIG. 16). These data indicate that the killing of MRSA persisters is achieved only when membrane damage by membrane-active agents is sufficiently severe to show increased membrane fluidity, potentially making membrane fluidity a biophysical indicator to identify anti-persister antimicrobials.

Discussion of Examples 1-8

Membrane-active agents have attractive properties as antimicrobial agents, including fast killing rates, anti-persister potency, synergism with other antibiotics, and a low probability of resistance selection. Unfortunately, most of these agents also indiscriminately disrupt mammalian membranes. However, bacteria and animals have different membrane lipid compositions, and evolution has taken advantage of these differences as reflected in the production of cationic antimicrobial peptides by animals and plants that specifically target bacterial cells. Gram-positive bacterial membrane lipid bilayers, including those of S. aureus, contain approximately 25% anionic phospholipids such as cardiolipin and phosphatidylglycerol. Conversely, mammalian membrane lipid bilayers are composed of zwitterionic (neutral) phospholipids and 20-50% cholesterol. Cationic antimicrobial peptides show a binding preference to negatively-charged bacterial membranes compared to neutrally-charged mammalian membranes. In addition, cholesterol embedded in mammalian membrane lipid bilayers creates a condensing effect that confers membrane rigidity and prevents the penetration of antimicrobial peptides.

The membrane selectivity of bithionol for Gram-positive bacterial membranes is likely due to differences in membrane lipid compositions between bacteria and mammals. The penetration of bithionol into negatively-charged bacterial mimetic lipid bilayers (7DOPC/3DOPG) is energetically favorable, whereas bithionol penetration into cholesterol-rich mammalian mimetic lipid bilayers (7POPC/3cholesterol) is energetically unfavorable (FIGS. 2A, B). Further, as the proportion of cholesterol increases from 0 to 30%, the penetration of bithionol becomes increasingly unfavorable (FIG. 10A), indicating that cholesterol plays a key role in bithionol's membrane selectivity.

S. aureus mutants displaying resistance to daptomycin or cationic antimicrobial peptides often show an increased net bacterial surface positive charge that creates electrostatic repulsion. Since bithionol is not positively charged, it is expected that bithionol will be effective against daptomycin-insensitive mutants. Indeed, bithionol has an MIC of 0.5 μg/mL against two daptomycin-intermediate resistant MRSA strains, BF2 and BF4, and MICs of 0.5—2 μg/mL against several daptomycin-intermediate or-resistant Enterococcal strains (FIG. 6).

Most membrane-active antimicrobials have amphipathic structures, including a lipophilic side chain and polar head group. Surprisingly, bithionol does not share these typical structural characteristics with conventional membrane-active antimicrobials. Instead, there are initial interactions between the hydrophilic head groups of the lipid bilayer and the phenols. As expected, these two polarized phenolic hydroxyl groups play a major role in the initial binding via hydrogen bonding to phospholipid headgroups. A methoxy analog of bithionol nullified bioactivity supporting the speculated mechanism of action (FIGS. 5, 7, and 13).

Although chlorine moieties seem to provide polar interactions with lipid headgroups, they also play a key role in lipid bilayer perturbation. Briefly, after attachment, the binding affinity of bithionol is dominated by hydrophobic interactions between its aromatic rings and the hydrophobic tails of the membrane lipids, which drives the penetration of the chlorinated benzene into the outer leaflet of the lipid bilayer to effect lipid bilayer perturbation (FIG. 9A, C). Accordingly, the replacement of chlorine with fluorine resulted in a decrease in antimicrobial potency (FIGS. 5 and 14). Replacement with the larger bromine atoms initially caused a disadvantage for initial binding to lipid bilayers with a positive energy barrier (FIG. 7), but caused increased destabilization of the membrane compared to smaller halogens after penetration. Collectively, the antimicrobial activity of bithionol can be modulated by binding affinity to lipid head groups, penetration depth, and molecule size.

All membrane-active compounds that kill MRSA persister cells permeabilize the persisters as measured by SYTOX Green staining. Although daptomycin is regarded to be a membrane-active antibiotic, it does not induce rapid SYTOX Green membrane permeabilization and accordingly does not kill MRSA persisters (Fig. IC and FIG. 8C). Daptomycin is known to form membrane pores in a calcium dependent manner. However, the pore formed by daptomycin selectively confers permeability to cations, such as Na⁺, K⁺, and alkali metal ions, but does not allow permeabilization of large molecules. On the other hand, not all compounds that permeabilize MRSA persisters kill them. Importantly, a compound-induced increase in membrane fluidity is required for the killing of MRSA persister cells. For example, brominated bithionol analogs, as well as nTZDpa and its analogs that exhibit anti-MRSA persister potency, induce both SYTOX Green membrane permeabilization and an increase in membrane fluidity (FIG. 5, FIG. 4, and FIGS. 13 and 14).

Insertion of compounds into membrane bilayers can increase membrane disorder and fluidity, which subsequently causes passive membrane permeabilization. However, for some membrane-active compounds, the initiation of SYTOX Green membrane permeabilization occurs at a lower concentration than is required to increase membrane fluidity (FIGS. 2D, E and FIGS. 13 and 14). Furthermore, some membrane-active agents, such as bitin-S, bithionol fluorine analogs, and nTZDpa-analogs 6 and 11 induce SYTOX Green membrane permeabilization, but do not cause the change in membrane fluidity (FIG. 4B and FIGS. 13 and 14). Bacterial membranes consist of lipid rafts organized into microdomains having different lipid compositions. Depending on lipid compositions, one domain may be less ordered and more fluid, while another domain may be more ordered and rigid. Because ordered and rigid domains show more resistance to membrane active agents, up to a certain threshold concentration, only less ordered domains would be affected by membrane-active compounds, thus making them SYTOX Green permeable. However, this type of localized membrane damage may not be sufficient to cause an overall increase in membrane fluidity. Over the threshold concentration, most membrane domains would be disrupted, and subsequently overall membrane fluidity would increase. It is also possible that some compounds may attack only less ordered and more fluid areas of membranes, which causes SYTOX Green membrane permeability, but not an overall increase in membrane fluidity. In any case, the killing of MRSA persisters is apparently only achieved when the bacterial membrane is sufficiently damaged to show increased membrane fluidity as detected by Laurdan GP.

In conclusion, the clinically approved anthelmintic bithionol and the newly synthesized exemplified compounds are effective antimicrobial agents against both multidrug-resistant and -persistent Gram-positive pathogens. Bithionol kills Gram-positive (but not Gram-negative) bacterial cells by disrupting lipid bilayers, while maintaining high selectivity for bacterial compared to mammalian membranes, a consequence of the presence of cholesterol in mammalian membranes. Further, bithionol in combination with gentamicin effectively eradicates S. aureus persisters and significantly reduces bacterial burden in a mouse model of chronic deep-seated MRSA infection. Increased membrane fluidity is a biophysical indicator to identify potent anti-persister compounds. This provides further understanding of the molecular mechanisms by which membrane-active small molecules selectively disrupt Gram-positive bacterial over mammalian membranes and support the conclusion that membrane-active antimicrobial agents have promising potential to be used for treating chronic infections caused by bacterial persisters.

### Example 9

### Preparation of Compound 1

Compound 1 was prepared by according to a synthetic scheme shown below using commercially available starting materials. In short, beginning with commercially available bithionol, treatment with N-Boc-2-bromoethylamine in the presence of base followed by subsequent Boc-deprotection afforded 1.

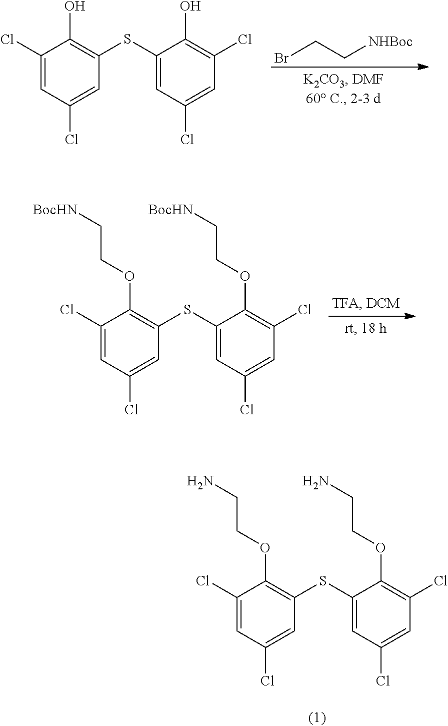 [SMILES: Oc1c(Cl)cc(Cl)cc1Sc1cc(Cl)cc(Cl)c1O ; CC(C)(C)OC(=O)NCCBr ; CC(C)(C)OC(=O)NCCOc1c(Cl)cc(Cl)cc1Sc1cc(Cl)cc(Cl)c1OCCNC(=O)OC(C)(C)C ; NCCOc1c(Cl)cc(Cl)cc1Sc1cc(Cl)cc(Cl)c1OCCN]

### Example 10

### Antibacterial Activity of Compound 1

Antimicrobial activity of each analog was tested against four MRSA strains (MW2, BF1, BF2, and BF3), Pseudomonas aeruginosa (PA14), Klebsiella pneumonia (WGLW2), and Acinetobacter baumannii (ATCC 17978). Results are shown in Table 1.

TABLE 1

| bacteria | MIC (μg/mL) |
|---|---|
| MRSA MW2 | 64 |
| MRSA BF1 | 64 |
| MRSA BF2 | 64 |
| MRSA BF3 | 32 |
| P. aeruginosa PA14 | >64 |
| K. pneumoniae WGLW2 | 64 |
| A. baumannii ATCC 17978 | 64 |

Additional experimental details are described in Cheng et al., Bioorganic & Medicinal Chemistry, 30 (9), 2020, which is incorporated herein by reference in its entirety.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 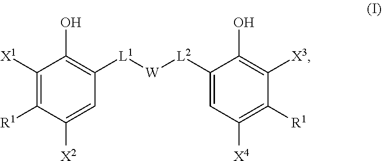
or a pharmaceutically acceptable salt thereof, wherein:
W is selected from S, S(═O), S(═O)₂, O, and C(═O);
L¹ is selected from C₁₋3 alkylene, C₂₋₄ alkenylene, C₂₋₄ alkynylene, and C₃₋₅ cycloalkylene, each of which is optionally substituted with 1, 2, or 3 substituents independently selected from OH, NH₂, NO₂, CN, and halo; or L¹ is absent;
L² is selected from C₁₋3 alkylene, C₂₋₄ alkenylene, C₂₋₄ alkynylene, and C₃₋₅ cycloalkylene, each of which is optionally substituted with 1, 2, or 3 substituents independently selected from OH, NH₂, NO₂, CN, and halo; or L² is absent;
X¹, X², X³, and X⁴ are each independently selected from halo, Cy^{A}, CN, NO₂, C₁₋₆ alkyl, C₁₋₆ haloalkyl, C₂₋₆ alkenyl, C₂₋₆ alkynyl, SR^{a1}, S(O)R^{b1}, S(O)₂R^{b1}, and OR^{a1}; wherein said C₁₋₆ alkyl, C₂₋₆ alkenyl, and C₂₋₆ alkynyl are each optionally substituted with 1, 2, or 3 substituents independently selected from Cy^{A}, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy^{A} is independently selected from C₆₋₁₀ aryl, C₃₋₁₀ cycloalkyl, 5-10 membered heteroaryl, and 4-10 membered heterocycloalkyl, each of which is optionally substituted with 1, 2, or 3 substituents independently selected from R^{c}Y;
each R^{Cy} is independently selected from halo, C₁₋₄ alkyl, C₁₋₄ haloalkyl, C₂₋₆ alkenyl, C₂₋₆ alkynyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{cl}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{cl}R^{d1}, S(O)₂R^{b1}, and S(O)₂NR^{c1}R^{d1};
R¹ and R² are each independently selected from H, halo, CN, NO₂, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from halo, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each R^{a1}, R^{b1}, R^{c1}, R^{d1}, R^{a2}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl are each optionally substituted with 1, 2, or 3 substituents independently selected from R^{g};
or any R^{c1} and R^{d1} together with the N atom to which they are attached form a 4-, 5-, 6-, or 7-membered heterocycloalkyl group optionally substituted with 1, 2, or 3 substituents independently selected from R^{g};
or any R^{c2} and R^{d2} together with the N atom to which they are attached form a 4-, 5-, 6-, or 7-membered heterocycloalkyl group optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO-C₁₋₃ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino;
provided that the compound of Formula (I) is not any one of the following compounds:

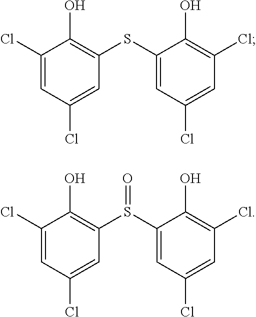

2. The compound of claim 1, wherein
W is selected from S, S(═O), and O;
L¹ is C₁₋3 alkylene or absent;
L² is C₁₋3 alkylene or absent;
X¹, X², X³, and X⁴ are each independently selected from halo, Cy^{A}, C₁₋₆ alkyl, and C₁₋₆ haloalkyl, wherein said C₁₋₆ alkyl is optionally substituted with a substituent selected from Cy^{A}, OH, NO₂, CN, halo, C₁₋₆ alkoxy, C₁₋₆ haloalkoxy, amino, C₁₋₆ alkylamino, and di(C₁₋₆ alkyl)amino;
each Cy^{A} is independently a phenyl, optionally substituted with 1 or 2 substituents independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino; and
R¹ and R² are each independently selected from H, halo, CN, NO₂, C₁₋₆ alkyl, C₁₋₆ haloalkyl, C₁₋₆ alkoxy, and C₁₋₆ haloalkoxy.

3. The compound of claim 2, wherein the compound of Formula (I) has formula: 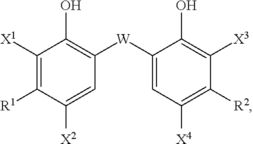
or a pharmaceutically acceptable salt thereof.

4. The compound of claim 3, wherein the compound of Formula (I) has formula: 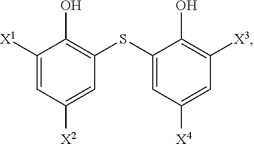
or a pharmaceutically acceptable salt thereof.

5. The compound of claim 4, wherein at least one of X¹, X², X³, and X⁴ is selected from Br, Cy^{A}, C₁₋₆ alkyl, and C₁₋₆ haloalkyl, wherein said C₁₋₆ alkyl is optionally substituted with a substituent selected from Cy^{A}, OH, NO₂, CN, halo, C₁₋₆ alkoxy, C₁₋₆ haloalkoxy, amino, C₁₋₆ alkylamino, and di(C₁₋₆ alkyl)amino.

6. The compound of claim 4, wherein:
X¹ and X³ are each independently Br or F, and
X² and X⁴ are each independently Cl, F, or Br.

7. The compound of claim 4, wherein:
X¹ and X³ are each independently Cl, F, or Br, and
X² and X⁴ are each independently Br or F.

8. The compound of claim 1, wherein the compound of Formula (I) is selected from any one of the following compounds: 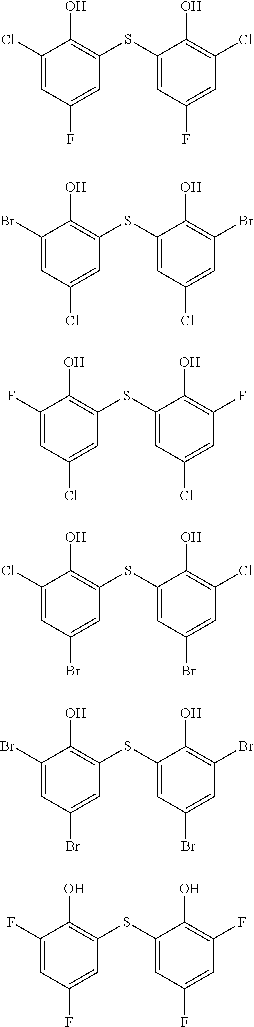
or a pharmaceutically acceptable salt thereof.

9. A pharmaceutical composition comprising a compound of claim 1, or a pharmaceutically acceptable salt thereof, and a pharmaceutically acceptable carrier.

10. A method of killing or inhibiting growth of a Gram-positive bacteria, the method comprising contacting the bacteria with an effective amount of a compound of claim 1, or a pharmaceutically acceptable salt thereof.

11. The method of claim 10, wherein the bacteria is tolerant or resistant to one or more other antibiotic agents.

12. The method of claim 11, wherein the bacteria is tolerant or resistant to one or more of the antibiotic agents selected from methicillin, vancomycin, rifampicin, gentamicin and ciprofloxacin.

13. The method of claim 11, wherein the bacteria is selected from S. aureus, methicillin-resistant S. aureus (MRSA), vancomycin-resistant S. aureus (VRSA), vancomycin-resistant Enterococcus (VRE), E. faecalis, E. faecium, B. subtilis, and B. anthracis.

14. A method of treating a bacterial infection caused by Gram-positive bacteria, the method comprising administering to the subject in need thereof a therapeutically effective amount of a compound of claim 1, or a pharmaceutically acceptable salt thereof.

15. The method of claim 14, wherein the bacterial infection is selected from atopic dermatitis, sinusitis, food poisoning, abscess, pneumonia, meningitis, osteomyelitis, endocarditis, bacteremia, sepsis, and urinary tract infection.

16. The method of claim 14, further comprising administering to the subject at least one additional antibiotic agent, or a pharmaceutically acceptable salt thereof

17. The method of claim 16, wherein the additional antibiotic agent is selected from gentamicin and nTZDpa, or a pharmaceutically acceptable salt thereof.

18. A method of killing or inhibiting growth of Gram-positive bacteria which is tolerant or resistant to one or more other antibiotic agents, the method comprising contacting the bacteria with an effective amount of a compound of formula: 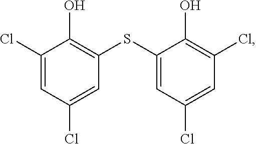 [SMILES: Oc1c(Cl)cc(Cl)cc1Sc1cc(Cl)cc(Cl)c1O]
or a pharmaceutically acceptable salt thereof.

19. (canceled)

20. A method of treating a bacterial infection caused by Gram-positive bacteria which is tolerant or resistant to one or more other antibiotic agents, the method comprising administering to the subject in need thereof a therapeutically effective amount of a compound of formula: 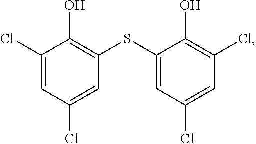 [SMILES: Oc1c(Cl)cc(Cl)cc1Sc1cc(Cl)cc(Cl)c1O]
or a pharmaceutically acceptable salt thereof.

21. A compound of formula (1): 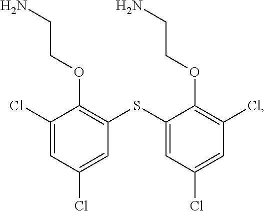 [SMILES: NCCOc1c(Cl)cc(Cl)cc1Sc1cc(Cl)cc(Cl)c1OCCN]
or a pharmaceutically acceptable salt thereof.

22.-25. (canceled)
